(19) United States
(12) Patent Application Publication
(10) Pub. No.: US 2009/0118350 A1
(43) Pub. Date: May 7, 2009

(54) Biomarkers for Identifying Efficacy of Tegaserod in Patients with Chronic Constipation

(76) Inventor: LeeAnne McLean, Medway, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 11/722,580

(22) PCT Filed: Jan. 3, 2006
(86) PCT No.: PCT/US06/00022

§ 371 Date: Jun. 22, 2007

Related U.S. Application Data
(60) Provisional application No. 60/641,238, filed on Jan. 4, 2005.

(51) Int. Cl. A61K 31/404; C07D 209/14; A61P 1/00; C12Q 1/68

(52) U.S. Cl. 514/415; 548/505; 435/6

(57) ABSTRACT

Pharmacogenetics was used to evaluate the effect of polymorphisms in select candidate genes on the response of patients with chronic constipation to tegaserod (Zelmac®/Zelnorm®). The analysis identified twelve single nucleotide polymorphisms (SNPs) in six genes (HTR4, HTR3B, MLN, AQP3, SLC12A2, SCNN1A) that were associated with at least a 60% response rate to tegaserod and an odds ratios of 5 or greater (compared to placebo) after 4 weeks of treatment. The identified genes display a wide range of different functions, including serotonin signaling, secretion and motility, all of which are important in maintaining the normal function of the gastrointestinal tract. Thus, these data imply that chronic constipation may result from a variety of pathophysiological mechanisms related to variants in the above identified genes, all of which respond well to treatment with tegaserod. Patients without these variants do not respond to treatment significantly more than they do to placebo, which could indicate that their chronic constipation is not due to pathophysiological mechanisms but rather to environmental or possibly psychological factors. Patients with these variants are also less likely to respond to placebo, again implying that these variants are associated with a true pathophysiology.

### FIELD OF THE INVENTION

This invention relates generally to the analytical testing of tissue samples in vitro, and more particularly to aspects of genetic polymorphisms for identifying individuals with chronic constipation that are more likely to respond to treatment with tegaserod.

### BACKGROUND OF THE INVENTION

The prevalence of chronic constipation is quite common, with most estimates ranging from 10-20% of an otherwise healthy population. Higgins & Johanson, Am. J. Gastroenterol. 99:750-9 (2004); Talley et al., Am. J. Gastroenterol. 98: 1107-11 (2003). In the United States alone, 2.5 million visits to physicians are due to constipation. Sonnenburg & Koch, Dig. Disc. Sci. 34: 606-11 (1989). There a number of subtypes of chronic constipation, including slow-transit constipation, pelvic floor dysfunction, functional constipation and constipation-predominant irritable bowel syndrome (C-IBS). Prather, Rev. Gastroenterol. Disorders, 4: S11-16 (2004). While symptoms may vary depending on the subtype, in general they include infrequent bowel movements (<3/week) associated with persistent symptoms of straining, hard or lumpy stools, and a feeling of incomplete evacuation. Thompson et al., Gut 45 (suppl II): 43-7 (1999).

Tegaserod (Zelnorm®/Zelmac®; HTF919) was approved by the United States Food and Drug Administration (FDA) in July 2002 for the short-term relief of constipation-predominant irritable bowel syndrome (C-IBS) and in August 2004 for the indication of chronic constipation. Tegaserod is an aminoguanidine indole compound that acts as an agonist of serotonin (5-HT₄) receptors (HTR4) and has been demonstrated to act as a promotile drug throughout the gastrointestinal (GI) tract in several animal models. Pfannkuche H J et al., Neurogastroenterol. Motil. 7: 280 (1995); Grider J R et al., Gastroenterology; 115:370-80 (1998). Tegaserod has also been shown to significantly increase colonic transit time in healthy volunteers (Degen L et al., Aliment Pharmacol. Ther. 15 (11):1745-51 (2001)), patients with IBS-C (Prather C M et al., Gastroenterology; 118 (3):463-8 (2000)), and patients with chronic constipation (Johanson et al., Clin. Gastroenterol. Hepatol. 2: 796-805 (2004)). In addition to its effects on intestinal motility, there is evidence that 5-HT₄ receptor agonists can have an effect on secretion (Kellum J M et al., Am. J. Physiol. 277: G515-G520 (1999)) as well as on visceral sensation in animal models (Schikowski A et al., Neurogastroenterol. Motil. 14: 221-227 (2002); Coelho A M et al., Gastroenterol. 118 (4, suppl. 2): A835; Yu S et al., Neurogastroenterol. Motil. 13: 445 (2001)).

However, not all patients respond to tegaserod significantly more than they do to placebo. A recent study of chronic constipation patients across North and South America demonstrated response rates of 43% to tegaserod compared with 25% to placebo. Johanson et al., Clin. Gastroenterol. Hepatol. 2: 796-805 (2004). There is currently no understanding of why some patients don't respond. Accordingly, there is a need in the art for additional insight into how tegaserod works. Furthermore, a deeper understanding of the importance of the identified genes in chronic constipation, or other gastrointestinal diseases, could lead to the development of novel therapies that are much more effective at treating gastrointestinal disorders.

### SUMMARY OF THE INVENTION

The invention answers these needs. The invention usefully provides a deeper understanding of how tegaserod works and provides more effective therapies for the treatment of chronic constipation. In one aspect, the invention shows that chronic constipation, in some patients, is a diagnostically identifiable disease. Chronic constipation may result from a variety of pathophysiological mechanisms related to variants in the several genes (HTR4, HTR3B, AQP3, MLN, SLC12A2, SCNN1A, TPH)— all of which respond well to treatment with tegaserod. Interestingly, patients with the diagnostically identifiable variants in the biomarker genes of the invention are also less likely to respond to placebo, again implying that these variants are associated with a true pathophysiology. In another aspect, the invention shows that some patients without these biomarker variants do not respond to treatment significantly more than they do to placebo, which could indicate that their chronic constipation is not due to physiological mechanisms but rather may be due to environmental or possibly psychological factors.

In one embodiment, the invention provides for the use of tegaserod the manufacture of a medicament for the treatment of chronic constipation in a selected patient population, wherein the patient population is selected on the basis of one or more biomarkers for the efficacy of tegaserod by the patients. These biomarkers are single nucleotide polymorphisms (SNPs) in six identified genes: the gene for 5-HT₄ receptor (HTR4; SEQ ID NOS:1-6); the gene for 5-HT₃ receptor, subunit B (HTR3B; SEQ ID NO:7); the gene for motilin (MLN; SEQ ID NO:8); the gene for aquaporin 3 water channel (AQP3; SEQ ID NO:9); the gene for solute carrier family 12, member A2, also known as the sodium/potassium/chloride cotransporter 1 (NKCC1) (SLC12A2; SEQ ID NOS:10-11); and the gene for non-voltage gated sodium channel, alpha subunit, also known as the amiloride-sensitive epithelial sodium channel, alpha subunit (ENaC alpha) (SCNN1A; SEQ ID NO:12). A total of 12 SNPs were identified across these six genes that were associated with higher response rates to tegaserod. The patients having a profile which includes one or more of these twelve SNPs indicative of the efficacy of tegaserod are identified as included in the selected “high responder” patient population.

In a different embodiment, the invention provides for the use of tegaserod the manufacture of a medicament for the treatment of chronic constipation in another selected patient population, wherein this patient population is selected on the basis of other biomarkers for the efficacy of tegaserod by the patients. In this embodiment, the markers are polymorphisms in six genes identified in the paragraph above, plus two polymorphisms in the gene for tryptophan hydroxylase 1 (TPH1; SEQ ID NOS:13-14). In this embodiment, the patients having a profile with one or more of these SNPs indicative of the efficacy of tegaserod are identified as included in the selected “high responder” patient population.

In another embodiment, the invention provides a method for identifying individuals with chronic constipation that are more likely to respond to treatment with tegaserod than to treatment with a placebo, based upon the SNP profiles in the biomarker genes of the individuals. In one embodiment, the individual is a vertebrate. In a particular embodiment, the vertebrate is a mammal. In a more particular embodiment, the mammal is a primate, such as a cynomolgus monkey or a human.

The invention also provides a method for treating chronic constipation in a subject. The method involves first obtaining the SNP profile of the subject in the biomarker genes of the invention. When the SNP profile of the subject in the biomarker genes of the invention is determined to be predictive of efficacy of treatment by tegaserod, then tegaserod is administered to the subject to treat the subject's chronic constipation. As used herein, the administration of an agent or drug to a subject or patient includes self-administration and the administration by another. Such an approach, which contains aspects of therapeutics and diagnostics, is termed “theranostic” by many of those of skill in the art.

The invention also provides methods for determining a subject for inclusion in a clinical trial for the treatment of gastrointestinal disorders by tegaserod, based upon an analysis of biomarkers expressed in the subject to be treated. The gastrointestinal disorders may include chronic constipation, constipation-predominant irritable bowel syndrome, functional dyspepsia, gastroesophogeal reflux disease, and diabetic gastropathy. The subject may be included in the clinical trial when its biomarker SNP profile suggests, due to comparison to known biomarker SNP profiles, that the subject may be susceptible to the appropriate tegaserod treatment regimen. Conversely, the subject may be excluded from the clinical trial when the subject's SNP profile is dissimilar to a profile indicative of efficacy of tegaserod treatment. Such similarities or dissimilarities are observable to those of skill in the art.

The invention also provides clinical assays, kits and reagents for determining treatment efficacy of a condition for which administration of tegaserod is indicated. In one embodiment, the kits contain reagents for determining the SNP profile of biomarker genes by hybridization. In another embodiment, the kits contain reagents for determining the SNP profile of biomarker genes by the polymerase chain reaction.

### BRIEF DESCRIPTION OF THE DRAWINGS

FIG. 1 is a bar graph showing the response rates as function of presence of “high responder” genotypes, after four weeks of treatment. Response=mean increase of one or more complete spontaneous bowel movements (CSBMs)/week over baseline; intent-to-treat (ITT) Caucasians with at least seven days of treatment; the twelve SNPs include the original twelve “high responder” SNPs; the fourteen SNPs includes the addition of the two TPH1 SNPs; (%) indicates percentage of genotyped population in each category.

FIG. 2 is a set of error bars showing the odds ratios as function of presence of “high responder” genotypes, after four weeks of treatment. The twelve SNPs include the original twelve “high responder” SNPs; the fourteen SNPs includes the addition of the two TPH1 SNPs; boxes represent the odds ratios and the lines represent the 95% confidence interval.

FIG. 3 is a drawing of the locations of SNPs genotyped across the HTR4 genomic region and determination of linkage disequilibrium. The upper panel shows that twelve SNPs were initially selected to span the majority of the genomic region of the HTR4 gene. The boxes indicate those SNPs that were associated with the higher than average response to tegaserod. Two SNP assays (SNP-3802 and SNP-3803) that did not yield good quality genotypes, and thus not utilized in evaluation of response, are highlighted. The lower panel shows the linkage disequilibrium (LD) between the ten good genotype assays for HTR4. The lower left quadrant indicates the degree of linkage disequilibrium between the SNPs (higher value indicates strong LD) and the upper right quadrant indicates the relative distance (in base pairs) between the SNPs. Note that the six SNPs that showed higher response to tegaserod (SNPs 1746, 3754, 3753, 3743, 3756, and 3747) show a high degree of linkage disequilibrium (>0.6) between each other, particular the first 4 of these SNPs (1746, 3754, 3753, and 3743), with LD>0.8.

FIG. 4 shows the structure of the 5-HT4 receptor, showing a schematic presentation of alternative splicing possibilities among the different 5-HT4 receptor exons indicated by connecting lines. Boxes represent exons, whereas introns are shown as bold lines. The dotted line between exon 4 and 5 represents the splicing that will include exon h into the mRNA, whereas the splice event following the dashed line omits exon h. Downstream of exon 5 the different combinations with C-terminal exons are depicted by using differently formatted lines for every splice variant. The C-terminal variants e and f are generated on the basis of two alternative splice acceptor sites within exon g.

### DETAILED DESCRIPTION OF THE PREFERRED EMBODIMENTS

Pharmacogenetics was used to evaluate the effect of polymorphisms in select candidate genes on the response of patients with chronic constipation to tegaserod (Zelmac®/Zelnorm®; HTF919). A total of 55 single nucleotide polymorphisms (SNPs) in twenty-three genes relating to the drug target (5-HT₄ receptor, HTR4), the serotonin pathway, solute transport, and gut motility were evaluated for patients enrolled in a clinical trial. Analysis was limited to Caucasians only in the intent-to-treat (ITT) population who had completed at least seven days of treatment with either high dose tegaserod (6 mg bid) or placebo. Results of analysis identified twelve SNPs in six genes [HTR4 (gene for 5-HT₄ receptor; SEQ ID NOS. 1-6; see SEQUENCE LISTING below), HTR3B (gene for 5-HT₃ receptor, subunit B; SEQ ID NO. 7), MLN (gene for motilin; SEQ ID NO:8), AQP3 (gene for aquaporin 3 water channel; SEQ ID NO:91), SLC12A2 (gene for solute carrier family 12, member A2; sodium/potassium/chloride cotransporter 1; NKCC1; SEQ ID NOS:10-11), SCNN1A (gene for non-voltage gated sodium channel, alpha subunit; ENaC alpha; SEQ ID NO:12)] that demonstrated at least a 60% response rate to tegaserod and an odds ratios of 5 or greater (compared to placebo) after 4 weeks of treatment. This was in comparison to the average overall response rates of 46% and 25% for tegaserod and placebo, respectively, with an odds ratio of 2.6. Using these twelve “high responder” SNPs as an embodiment, we found that individuals that at least one of the twelve SNPs (approximately 50% of the population) had a significantly higher response to tegaserod compared to placebo (62% vs. 23%, odds ratio=5.4). This is in contrast to those patients with none of the twelve SNPs who showed no significant difference in response to tegaserod compared to placebo (31% vs. 27%, odds ratio=1.3).

A second fourteen SNP embodiment, which included an additional two SNPs in TPH1 (Tryptophan hydroxylase 1; SEQ ID NOS: 13-14; see SEQUENCE LISTING below), showed that the 32% of the population who did not have any of these fourteen SNPs had no significantly different response to tegaserod compared to placebo (32% vs. 30% response, odds ratio=1.1). The 68% of the population that did have at least one of these fourteen SNPs demonstrated significantly higher response to tegaserod compared to placebo (53% vs. 22%, odds ratio=4.1), although to a lesser extent than what was seen using the fourteen SNP embodiment.

Based on these findings, chronic constipation results from a variety of pathophysiological mechanisms related to variants in the above identified genes, all of which respond well to treatment with tegaserod. Patients without these variants do not respond to treatment significantly more than they do to placebo, which could indicate that their chronic constipation is not due to pathophysiological mechanisms but rather to environmental or possibly psychological factors. Interestingly, patients with these variants are also less likely to respond to placebo, again implying that these variants are associated with a true pathophysiology.

The polymorphisms identified here may also be of relevance for other gastrointestinal disorders, such as constipation-predominant IBS, functional dyspepsia, gastroesophogeal reflux disease, and diabetic gastropathy.

Definitions. As used herein, “medical condition” includes but is not limited to any condition or disease manifested as one or more physical and/or psychological symptoms for which treatment is desirable, and includes previously and newly identified diseases and other disorders.

The term “clinical response” means any or all of the following: a quantitative measure of the response, no response, and adverse response (i.e., side effects).

In order to deduce a correlation between a clinical response to a treatment and a genotype or haplotype, genotype or haplotype data is obtained on the clinical responses exhibited by a population of individuals who received the treatment, hereinafter the “clinical population”. This clinical data may be obtained by analyzing the results of a clinical trial that has already been run and/or by designing and carrying out one or more new clinical trials.

The term “clinical trial” means any research study designed to collect clinical data on responses to a particular treatment, and includes but is not limited to phase I, phase II and phase III clinical trials. Standard methods are used to define the patient population and to enroll subjects.

It is preferred that the individuals included in the clinical population be graded for the existence of the medical condition of interest. This grading of potential patients could employ a standard physical exam or one or more lab tests. Alternatively, grading of patients could use haplotyping for situations where there is a strong correlation between haplotype pair and disease susceptibility or severity.

The therapeutic treatment of interest is administered to each individual in the trial population, and each individual's response to the treatment is measured using one or more predetermined criteria. It is contemplated that in many cases, the trial population will exhibit a range of responses, and that the investigator will choose the number of responder groups (e.g., low, medium, high) made up by the various responses. In addition, the gene for each individual in the trial population is genotyped and/or haplotyped, which may be done before or after administering the treatment.

As used herein, a “SNP nucleic acid” is a nucleic acid sequence, which comprises a nucleotide that is variable within an otherwise identical nucleotide sequence between individuals or groups of individuals, thus, existing as alleles. Such SNP nucleic acids are preferably from about 15 to about 500 nucleotides in length. The SNP nucleic acids may be part of a chromosome, or they may be an exact copy of a part of a chromosome, e.g., by amplification of such a part of a chromosome through PCR or through cloning. The SNP nucleic acids are referred to hereafter simply as “SNPs”. The SNP probes according to the invention are oligonucleotides that are complementary to an SNP nucleic acid.

The term “complementary” means complementary throughout the length of the oligonucleotide in the Watson and Crick sense of the word.

As used herein the term “polymorphism” shall mean any sequence variant present at a frequency of >1% in a population. The sequence variant may be present at a frequency significantly greater than 1% such as 5% or 10% or more. Also, the term may be used to refer to the sequence variation observed in an individual at a polymorphic site. Polymorphisms include nucleotide substitutions, insertions, deletions and microsatellites and may, but need not, result in detectable differences in gene expression or protein function.

The term “genotype” shall mean an unphased 5′ to 3′ sequence of nucleotide pairs found at one or more polymorphic sites in a locus on a pair of homologous chromosomes in an individual. As used herein, genotype includes a full-genotype and/or a sub-genotype.

The term “polynucleotide” shall mean any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein the term “gene” shall mean a segment of DNA that contains all the information for the regulated biosynthesis of an RNA product, including promoters, exons, introns, and other untranslated regions that control expression.

As used herein the term “locus” shall mean a location on a chromosome or DNA molecule corresponding to a gene or a physical or phenotypic feature.

As used herein the term “polypeptide” shall mean any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

The term “polymorphic site” shall mean a position within a locus at which at least two alternative sequences are found in a population, the most frequent of which has a frequency of no more than 99%.

The term “phased” means, when applied to a sequence of nucleotide pairs for two or more polymorphic sites in a locus, the combination of nucleotides present at those polymorphic sites on a single copy of the locus is known.

Single Nucleotide Polymorphisms. Sequence variation in the human genome consists primarily of single nucleotide polymorphisms (“SNPs”), the remainder being short tandem repeats (including micro-satellites), long tandem repeats (mini-satellites), and other insertions and deletions. An SNP is the occurrence of nucleotide variability at a single position in the genome, in which two alternative bases occur at appreciable frequency (i.e. >1%) in the human population. An SNP may occur within a gene or within intergenic regions of the genome.

An SNP is said to be “allelic” in that due to the existence of the polymorphism, some members of a species may have an unmutated sequence (i.e., the original allele) whereas other members may have a mutated sequence (i.e., the variant or mutant allele). In the simplest case, only one mutated sequence may exist, and the polymorphism is said to be diallelic. The occurrence of alternative mutations can give rise to triallelic polymorphisms, etc. SNPs are widespread throughout the genome, and SNPs that alter the function of a gene may be direct contributors to phenotypic variation. Due to their prevalence and widespread nature, SNPs have the potential to be important tools for locating genes that are involved in human disease conditions. See, e.g., Wang et al., Science 280: 1077-1082 (1998), which discloses a pilot study in which 2,227 SNPs were mapped over a 2.3 megabase region of DNA.

An association between a single nucleotide polymorphisms and a particular phenotype does not necessarily indicate or require that the SNP is causative of the phenotype. Instead, the association may merely be due to genome proximity between an SNP and those genetic factors actually responsible for a given phenotype, such that the SNP and said genetic factors are often observed together. Thus, an SNP may be in linkage disequilibrium (LD) with the “true” functional variant. LD, also known as allelic association, exists when alleles at two distinct locations of the genome are more highly associated than expected.

Thus an SNP may serve as a marker that has value by virtue of its proximity to a mutation that causes a particular phenotype.

SNPs that are associated with disorders may also have a direct effect on the function of the genes in which they are located. A sequence variant may result in an amino acid change or may alter exon-intron splicing, thereby directly modifying the relevant protein, or it may exist in a regulatory region, altering the cycle of expression or the stability of the mRNA. See, e.g., Nowotny et al., Current Opinions in Neuobiology 11:637-641 (2001).

It is increasingly clear that the risk of developing many common disorders and the metabolism of medications used to treat these conditions are substantially influenced by underlying genomic variations, although the effects of any one variant might be small. Therefore, an association between an SNP and a clinical phenotype suggests (1) the SNP may be functionally responsible for the phenotype, or (2) there may be other mutations near the location of the SNP on the genome that cause the phenotype. The 2^{nd} possibility is based on the biology of inheritance, in that large pieces of DNA are inherited, and markers may be in linkage disequilibrium (LD) due to their proximity and a lack of sufficiently separating recombination events.

Identification and characterization of SNPs. Many different techniques can be used to identify and characterize SNPs, including single-strand conformation polymorphism (SSCP) analysis, heteroduplex analysis by denaturing high-performance liquid chromatography (DHPLC), direct DNA sequencing and computational methods (see Shi et al., Clin. Chem. 47:164-172 (2001). Thanks to the wealth of sequence information in public databases, computational tools can be used to identify SNPs in silico by aligning independently submitted sequences for a given gene (either cDNA or genomic sequences). Cox et al., Hum Mutal 2001, 17:141-150.

The most common SNP typing methods currently include hybridization, primer extension, and cleavage methods. Each of these methods must be connected to an appropriate detection system. Detection technologies include fluorescent polarization, (see Chan et al., Genome Res. 9:492-499 (1999)), luminometric detection of pyrophosphate release (pyrosequencing), (see Ahmadiian et al., Anal. Biochem. 280:103-10 (2000)), fluorescence resonance energy transfer (FRET)-based cleavage assays, DHPLC, and mass spectrometry (see Shi, Clin. Chem. 47:164-172 (2001) and U.S. Pat. No. 6,300,076 B1). Other methods of detecting and characterizing SNPs are those disclosed in U.S. Pat. Nos. 6,297,018 B1 and 6,300,063 B1. The disclosures of the above references are incorporated herein by reference in their entirety.

In a particularly preferred embodiment, the detection of polymorphisms can be accomplished by means of INVADER™ technology (available from Third Wave Technologies Inc. Madison, Wis., USA). In this assay, a specific upstream “invader” oligonucleotide and a partially overlapping downstream probe together form a specific structure when bound to complementary DNA template. This structure is recognized and cut at a specific site by the Cleavase enzyme, resulting in the release of the 5′ flap of the probe oligonucleotide. This fragment then serves as the “invader” oligonucleotide with respect to synthetic secondary targets and secondary fluorescently labelled signal probes contained in the reaction mixture. This results in specific cleavage of the secondary signal probes by the Cleavase enzyme. Fluorescent signal is generated when this secondary probe, labelled with dye molecules capable of fluorescence resonance energy transfer, is cleaved. Cleavases have stringent requirements relative to the structure formed by the overlapping DNA sequences or flaps and can, therefore, be used to specifically detect single base pair mismatches immediately upstream of the cleavage site on the downstream DNA strand. See Ryan D et al., Molecular Diagnosis 4(2): 135-144 (1999) and Lyamichev V et al. Nature Biotechnology 17: 292-296 (1999), see also U.S. Pat. Nos. 5,846,717 and 6,001,567, the disclosures of which are incorporated herein by reference in their entirety.

Genotyping oligonucleotides of the invention are employed in practicing SNP typing methods such as those described above. Some embodiments of the invention contain two or more differently labelled genotyping oligonucleotides, for simultaneously probing the identity of nucleotides at two or more polymorphic sites. It is also contemplated that primer compositions may contain two or more sets of allele-specific primer pairs to allow simultaneous targeting and amplification of two or more regions containing a polymorphic site.

Genotyping oligonucleotides of the invention may be immobilized on or synthesized on a solid surface such as a microchip, bead, or glass slide (see, e.g., WO 98/20020 and WO 98/20019). Such immobilized genotyping oligonucleotides may be used in a variety of polymorphism detection assays, including but not limited to probe hybridization and polymerase extension assays. Immobilized genotyping oligonucleotides of the invention may comprise an ordered array of oligonucleotides designed to rapidly screen a DNA sample for polymorphisms in multiple genes at the same time.

An allele-specific oligonucleotide primer of the invention has a 3′ terminal nucleotide, or preferably a 3′ penultimate nucleotide, that is complementary to only one nucleotide of a particular SNP, thereby acting as a primer for polymerase-mediated extension only if the allele containing that nucleotide is present. Allele-specific oligonucleotide (ASO) primers hybridizing to either the coding or noncoding strand are contemplated by the invention. An ASO primer for detecting gene polymorphisms can be developed using techniques known to those of skill in the art.

Other genotyping oligonucleotides of the invention hybridize to a target region located one to several nucleotides downstream of one of the polymorphic sites identified herein. Such oligonucleotides are useful in polymerase-mediated primer extension methods for detecting one of the polymorphisms described herein and therefore such genotyping oligonucleotides are referred to herein as “primer-extension oligonucleotides.” In a preferred embodiment, the 3′-terminus of a primer-extension oligonucleotide is a deoxynucleotide complementary to the nucleotide located immediately adjacent to the polymorphic site.

Genotyping and Haplotyping. Oligonucleotide compositions and kits of the invention are useful in methods for genotyping and/or haplotyping a gene in an individual. The terms “genotype” and “haplotype” mean the genotype or haplotype containing the nucleotide pair or nucleotide, respectively, that is present at one or more of the polymorphic sites described herein, and may optionally also include the nucleotide pair or nucleotide present at one or more additional polymorphic sites in the gene. The additional polymorphic sites may be currently known polymorphic sites or sites that are subsequently discovered.

One embodiment of a genotyping method of the invention involves isolating from an individual a nucleic acid mixture comprising the two copies of a gene of interest or fragment thereof, and determining the identity of the nucleotide pair at one or more of the polymorphic sites in the two copies. As will be readily understood by the skilled artisan, the two “copies” of a gene in an individual may be the same allele or may be different alleles. In a particularly preferred embodiment, the genotyping method comprises determining the identity of the nucleotide pair at each polymorphic site.

Typically, the nucleic acid mixture is isolated from a biological sample taken from the individual, such as a blood sample or tissue sample. Suitable tissue samples include whole blood, semen, saliva, tears, urine, fecal material, sweat, buccal smears, skin and hair. The nucleic acid mixture may be comprised of genomic DNA, mRNA, or cDNA and, in the latter two cases, the biological sample must be obtained from an organ in which the gene is expressed. Furthermore, it will be understood by the skilled artisan that mRNA or cDNA preparations would not be used to detect polymorphisms located in introns or in 5′ and 3′ nontranscribed regions. If a gene fragment is isolated, it must contain the polymorphic sites to be genotyped.

One embodiment of the haplotyping method of the invention comprises isolating from an individual a nucleic acid molecule containing only one of the two copies of a gene of interest, or a fragment thereof, and determining the identity of the nucleotide at one or more of the polymorphic sites in that copy. The nucleic acid may be isolated using any method capable of separating the two copies of the gene or fragment. As will be readily appreciated by those skilled in the art, any individual clone will only provide haplotype information on one of the two gene copies present in an individual. If haplotype information is desired for the individual's other copy, additional clones will need to be examined. Typically, at least five clones should be examined to have more than a 90% probability of haplotyping both copies of the gene in an individual. In a particularly preferred embodiment, the nucleotide at each polymorphic site is identified.

In a preferred embodiment, a haplotype pair is determined for an individual by identifying the phased sequence of nucleotides at one or more of the polymorphic sites in each copy of the gene that is present in the individual. In a particularly preferred embodiment, the haplotyping method comprises identifying the phased sequence of nucleotides at each polymorphic site in each copy of the gene. When haplotyping both copies of the gene, the identifying step is preferably performed with each copy of the gene being placed in separate containers. However, if the two copies are labelled with different tags, or are otherwise separately distinguishable or identifiable, it is possible in some cases to perform the method in the same container. For example, if the first and second copies of the gene are labelled with different first and second fluorescent dyes, respectively, and an allele-specific oligonucleotide labelled with yet a third different fluorescent dye is used to assay the polymorphic sites, then detecting a combination of the first and third dyes would identify the polymorphism in the first gene copy, while detecting a combination of the second and third dyes would identify the polymorphism in the second gene copy.

In both the genotyping and haplotyping methods, the identity of a nucleotide (or nucleotide pair) at a polymorphic sites may be determined by amplifying a target regions containing the polymorphic sites directly from one or both copies of the gene, or fragments thereof, and sequencing the amplified regions by conventional methods. It will be readily appreciated by the skilled artisan that only one nucleotide will be detected at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where an SNP is known to be guanine and cytosine in a reference population, a site may be positively determined to be either guanine or cytosine for all individuals homozygous at that site, or both guanine and cytosine, if the individual is heterozygous at that site. Alternatively, the site may be negatively determined to be not guanine (and thus cytosine/cytosine) or not cytosine (and thus guanine/guanine).

In addition, the identity of the alleles present at any of the polymorphic sites of the invention may be indirectly determined by genotyping other polymorphic sites in linkage disequilibrium with those sites of interest. As described supra, two sites are said to be in linkage disequilibrium if the presence of a particular variant at one site is indicative of the presence of another variant at a second site. See, Stevens, J C, Mol. Diag. 4:309-317 (1999)). Polymorphic sites in linkage disequilibrium with the polymorphic sites of the invention may be located in regions of the same gene or in other genomic regions. Genotyping a polymorphic site in linkage disequilibrium with the novel polymorphic sites described herein may be performed by, but is not limited to, any of the above-mentioned methods for detecting the identity of the allele at a polymorphic site.

The target regions may be amplified using any oligonucleotide-directed amplification method, including but not limited to polymerase chain reaction (PCR) (U.S. Pat. No. 4,965,188), ligase chain reaction (LCR) (Barany et al., Proc. Natl. Acad. Sci. USA 88:189-193 (1991); published PCT patent application WO 90/01069), and oligonucleotide ligation assay (OLA) (Landegren et al., Science 241:1077-1080 (1988)). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

Other known nucleic acid amplification procedures may be used to amplify the target region, including transcription-based amplification systems (U.S. Pat. No. 5,130,238; EP 329,822; U.S. Pat. No. 5,169,766, published PCT patent application WO 89/06700) and isothermal methods (Walker et al., Proc. Natl. Acad. Sci. USA 89:392-396, 1992).

A polymorphism in the target region may be assayed before or after amplification using one of several hybridization-based methods known in the art. Typically, allele-specific oligonucleotides are utilized in performing such methods. The allele-specific oligonucleotides may be used as differently labelled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs. Preferably, the members of the set have melting temperatures within 5° C., and more preferably within 2° C., of each other when hybridizing to each of the polymorphic sites being detected.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking, baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibres, chips, dishes, and beads. The solid support may be treated, coated or derivatized to facilitate the immobilization of the allele-specific oligonucleotide or target nucleic acid.

The genotype or haplotype for the gene of an individual may also be determined by hybridization of a nucleic sample containing one or both copies of the gene to nucleic acid arrays and subarrays such as described in WO 95/11995. The arrays would contain a battery of allele-specific oligonucleotides representing each of the polymorphic sites to be included in the genotype or haplotype.

The identity of polymorphisms may also be determined using a mismatch detection technique, including but not limited to the RNase protection method using riboprobes (Winter et al., Proc. Natl. Acad. Sci. USA 82:7575 (1985); Meyers et al., Science 230:1242 (1985)) and proteins which recognize nucleotide mismatches, such as the E. coli mutS protein (Modrich P. Ann. Rev. Genet. 25:229-253 (1991)). Alternatively, variant alleles can be identified by single strand conformation polymorphism (SSCP) analysis (Orita et al., Genomics 5:874-879 (1989); Humphries et al., in Molecular Diagnosis of Genetic Diseases, R. Elles, ed., pp. 321-340 (1996)) or denaturing gradient gel electrophoresis (DGGE) (Wartell et al., Nucl. Acids Res. 18:2699-2706, (1990); Sheffield et al., Proc. Natl. Acad. Sci. USA 86:232-236 (1989)).

A polymerase-mediated primer extension method may also be used to identify the polymorphisms. Several such methods have been described in the patent and scientific literature and include the “Genetic Bit Analysis” method (WO 92/15712) and the ligase/polymerase mediated genetic bit analysis (U.S. Pat. No. 5,679,524). Related methods are disclosed in WO 91/02087, WO 90/09455, WO 95/17676, and U.S. Pat. Nos. 5,302,509 and 5,945,283. Extended primers containing a polymorphism may be detected by mass spectrometry as described in U.S. Pat. No. 5,605,798. Another primer extension method is allele-specific PCR. See, Ruafio et al., Nucl. Acids Res. 17:8392 (1989); Ruafio et al., Nucl. Acids Res. 19, 6877-6882 (1991); WO 93/22456; Turki et al., J. Clin. Invest. 95:1635-1641 (1995)). In addition, multiple polymorphic sites may be investigated by simultaneously amplifying multiple regions of the nucleic acid using sets of allele-specific primers as described in WO 89/10414.

In another aspect, the invention provides SNP probes, which are useful in classifying subjects according to their types of genetic variation. The SNP probes according to the invention are oligonucleotides, which discriminate between SNPs in conventional allelic discrimination assays.

In certain preferred embodiments, the oligonucleotides according to this aspect of the invention are complementary to one allele of the SNP nucleic acid, but not to any other allele of the SNP nucleic acid. Oligonucleotides according to this embodiment of the invention can discriminate between SNPs in various ways. For example, under stringent hybridization conditions, an oligonucleotide of appropriate length will hybridize to one SNP, but not to any other. The oligonucleotide may be labelled by a radiolabel or a fluorescent label. Alternatively, an oligonucleotide of appropriate length can be used as a primer for PCR, wherein the 3′ terminal nucleotide is complementary to one allele containing an SNP, but not to any other allele. In this embodiment, the presence or absence of amplification by PCR determines the haplotype of the SNP.

Genomic and cDNA fragments of the invention comprise at least one novel polymorphic site identified herein, have a length of at least 10 nucleotides, and may range up to the full length of the gene. Preferably, a fragment according to the present invention is between 100 and 3000 nucleotides in length, and more preferably between 200 and 2000 nucleotides in length, and most preferably between 500 and 1000 nucleotides in length.

In describing the polymorphic sites of the invention, reference is made to the sense strand of the gene for convenience. However, as recognized by the skilled artisan, nucleic acid molecules containing the gene may be complementary double stranded molecules and thus reference to a particular site on the sense strand refers as well to the corresponding site on the complementary antisense strand. Thus, reference may be made to the same polymorphic site on either strand and an oligonucleotide may be designed to hybridize specifically to either strand at a target region containing the polymorphic site. Thus, the invention also includes single-stranded polynucleotides that are complementary to the sense strand of the genomic variants described herein.

Determining Population Genotypes and Haplotypes. The present invention provides a method for determining the frequency of a given genotype or haplotype in a population. The method comprises determining the genotype or the haplotype for a gene present in each member of the population, wherein the genotype or haplotype comprises a nucleotide pair or nucleotide detected at one or more of the polymorphic sites in the gene, and calculating the frequency at which the genotype or haplotype is found in the population. The population may be a reference population, a family population, a same sex population, a population group, or a trait population (e.g., a group of individuals exhibiting a trait of interest such as a medical condition or response to a therapeutic treatment).

In another aspect of the invention, frequency data for genotypes and/or haplotypes found in a reference population are used in a method for identifying an association between a trait and a genotype or a haplotype. The trait may be any detectable phenotype, including but not limited to susceptibility to a disease or response to a treatment. The method involves obtaining data on the frequency of the genotypes or haplotypes of interest in a reference population and comparing the data to the frequency of the genotypes or haplotypes in a population exhibiting the trait. Frequency data for one or both of the reference and trait populations may be obtained by genotyping or haplotyping each individual in the populations using one of the methods described above. The haplotypes for the trait population may be determined directly or, alternatively, by the predictive genotype to haplotype approach described above.

In a preferred embodiment of the method, the trait is susceptibility to a disease, severity of a disease, the staging of a disease or response to a drug. Such methods have applicability in developing diagnostic tests and therapeutic treatments for all pharmacogenetic applications where there is the potential for an association between a genotype and a treatment outcome, including efficacy measurements, pharmacokinetic measurements and side effect measurements.

In another preferred embodiment of the method, the trait of interest is a clinical response exhibited by a patient to some therapeutic treatment, for example, response to a drug targeting or to a therapeutic treatment for a medical condition.

In another embodiment, the frequency data for the reference and/or trait populations are obtained by accessing previously determined frequency data, which may be in written or electronic form. For example, the frequency data may be present in a database that is accessible by a computer. Once the frequency data are obtained, the frequencies of the genotypes or haplotypes of interest in the reference and trait populations are compared. In a preferred embodiment, the frequencies of all genotypes and/or haplotypes observed in the populations are compared. If a particular genotype or haplotype for the gene is more frequent in the trait population than in the reference population at a statistically significant amount, then the trait is predicted to be associated with that genotype or haplotype.

In a preferred embodiment, the haplotype frequency data for different ethnogeographic groups are examined to determine whether they are consistent with Hardy-Weinberg equilibrium. D. L. Hartl et al., Principles of Population Genoinics, 3rd Ed. (Sinauer Associates, Sunderland, M A, 1997). Hardy-Weinberg equilibrium postulates that the frequency of finding the haplotype pair H₁/H₂ is equal to P_{H-W} (H₁/H₂)=2p(H₁) p (H₂) if H₁≠H₂ and P_{H-W} (H₁/H₂)=p (H₁) p (H₂) if H₁=H₂. A statistically significant difference between the observed and expected haplotype frequencies could be due to one or more factors including significant inbreeding in the population group, strong selective pressure on the gene, sampling bias, and/or errors in the genotyping process. If large deviations from Hardy-Weinberg equilibrium are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method such as, for example, CLASPER System™ technology (U.S. Pat. No. 5,866,404), SMD, or allele-specific long-range PCR (Michalotos-Beloin et al., Nucl Acids Res 24:4841-4843 (1996)).

In one embodiment of this method for predicting a haplotype pair, the method of the invention involves performing the following analysis. First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair. In rare cases, either no haplotypes in the reference population are consistent with the possible haplotype pairs, or alternatively, multiple reference haplotype pairs are consistent with the possible haplotype pairs. In such cases, the individual is preferably haplotyped using a direct molecular haplotyping method such as, for example, those discussed supra.

In a preferred embodiment, statistical analysis is performed by the use of standard analysis of variation (ANOVA) tests with a Bonferoni correction and/or a bootstrapping method that simulates the genotype/phenotype correlation many times and calculates a significance value. When many polymorphisms are being analyzed, a calculation may be performed to correct for a significant association that might be found by chance. For statistical methods for use in the methods of this invention, see: Statistical Methods in Biology, 3^{rd} edition, Bailey N T J, (Cambridge Univ. Press, 1997); Introduction to Computational Biology, Waterman M S (CRC Press, 2000) and Bioinformatics, Baxevanis A D & Ouellette B F F editors (John Wiley & Sons, Inc., 2001).

In another embodiment of the invention, a detectable genotype or haplotype that is in linkage disequilibrium with a genotype or haplotype of interest may be used as a surrogate marker. A genotype that is in linkage disequilibrium with another genotype may be discovered by determining if a particular genotype or haplotype for a given gene is more frequent in the population that also demonstrates the potential surrogate marker genotype than in the reference population. If the frequency is statistically significant, then the marker genotype is predictive of that genotype or haplotype, and can be used as a surrogate marker.

Correlation Between Genotype or Haplotype and Subject Response to Treatment. After both the clinical and polymorphism data have been obtained from a given subject, correlations between individual response and genotype or haplotype content are created. Correlations may be produced in several ways: in one method, individuals are grouped by their genotype or haplotype (or haplotype pair) (also referred to as a polymorphism group), and then the averages and standard deviations of clinical responses exhibited by the members of each polymorphism group are calculated.

In order to deduce a correlation between clinical response to a treatment and a genotype or haplotype, it is necessary to obtain data on the clinical responses exhibited by a population of individuals who received the treatment, hereinafter the “clinical population”. This clinical data may be obtained by analyzing the results of a clinical trial that has already been run and/or the clinical data may be obtained by designing and carrying out one or more new clinical trials.

These results are then analyzed to determine if any observed variation in clinical response between polymorphism groups is statistically significant. Statistical analysis methods which may be used are described in L. D. Fisher & G. vanBelle, Biostatistics: A Methiodology for the Health Sciences (Wiley-Interscience, New York, 1993). This analysis may also include a regression calculation of which polymorphic sites in the gene contribute most significantly to the differences in phenotype.

A second method for finding correlations between haplotype content and clinical responses uses predictive models based on error-minimizing optimization algorithms, one of which is a genetic algorithm (R. Judson, Genetic Algorithms and Their Uses in Chemistry in Reviews in Computational Chemistry, Vol. 10, pp. 1-73, K. B. Lipkowitz and D. B. Boyd, eds. (VCH Publishers, New York, 1997). Simulated annealing (Press et al., Numerical Recipes in C: The Art of Scientific Computing, Ch. 10 (Cambridge University Press, Cambridge) 1992), neural networks (E. Rich & K. Knight, Artificial Intelligence, 2nd Edition, Ch. 10 (McGraw-Hill, New York, 1991), standard gradient descent methods (Press et al., supra Ch. 10), or other global or local optimization approaches (see discussion in Judson, supra) can also be used.

Correlations may also be analyzed using analysis of variation (ANOVA) techniques to determine how much of the variation in the clinical data is explained by different subsets of the polymorphic sites in the gene. ANOVA is used to test hypotheses about whether a response variable is caused by or correlates with one or more traits or variables that can be measured (Fisher & vanBelle, supra, Ch. 10). In the EXAMPLE provided herein, the primary statistical analyses in this particular study were done using Mantel-Haenzel tests to determine odds ratios and 95% confidence limits, logistic regression, ANCOVA, and Fisher's Exact tests.

From the analyses described above, a mathematical model may be readily constructed by the skilled artisan that predicts clinical response as a function of genotype or haplotype content.

The identification of an association between a clinical response and a genotype or haplotype (or haplotype pair) for the gene may be the basis for designing a diagnostic method to determine those individuals who will or will not respond to the treatment, or alternatively, will respond at a lower level and thus may require more treatment, i.e., a greater dose of a drug. These diagnostic methods of the invention may take one of several forms: for example, a direct DNA test (i.e., genotyping or haplotyping one or more of the polymorphic sites in the gene), a serological test, or a physical exam measurement. The only requirement is that there be a good correlation between the diagnostic test results and the underlying genotype or haplotype. In a preferred embodiment, this diagnostic method uses the predictive haplotyping method described above.

A computer may implement any or all analytical and mathematical operations involved in practicing the methods of the present invention. In addition, the computer may execute a program that generates views (or screens) displayed on a display device and with which the user can interact to view and analyze large amounts of information relating to the gene and its genomic variation, including chromosome location, gene structure, and gene family, gene expression data, polymorphism data, genetic sequence data, and clinical population data (e.g., data on ethnogeographic origin, clinical responses, genotypes, and haplotypes for one or more populations). The polymorphism data described herein may be stored as part of a relational database (e.g., an instance of an Oracle database or a set of ASCII flat files). These polymorphism data may be stored on the computer's hard drive or may, for example, be stored on a CD-ROM or on one or more other storage devices accessible by the computer. For example, the data may be stored on one or more databases in communication with the computer via a network.

In other embodiments, the invention provides methods, compositions, and kits for haplotyping and/or genotyping the gene in an individual. The compositions contain oligonucleotide probes and primers designed to specifically hybridize to one or more target regions containing, or that are adjacent to, a polymorphic site. The methods and compositions for establishing the genotype or haplotype of an individual at the novel polymorphic sites described herein are useful for studying the effect of the polymorphisms in the etiology of diseases affected by the expression and function of the protein, studying the efficacy of drug targeting, predicting individual susceptibility to diseases affected by the expression and function of the protein, and predicting individual responsiveness to drugs targeting the gene product.

The invention also provides a computer system for storing and displaying polymorphism data determined for the gene. The computer system comprises a computer processing unit, a display, and a database containing the polymorphism data. The polymorphism data includes the polymorphisms, the genotypes and the haplotypes identified for a given gene in a reference population. In a preferred embodiment, the computer system is capable of producing a display showing haplotypes organized according to their evolutionary relationships.

Assessing Expression Levels. RT-PCR (real-time quantitative PCR) is one way to assess gene expression levels, e.g., of genes of the invention (e.g., those containing SNPs and polymorphisms of interest). The RT-PCR assay utilizes an RNA reverse transcriptase to catalyze the synthesis of a DNA strand from an RNA strand, including an mRNA strand. The resultant DNA may be specifically detected and quantified and this process may be used to determine the levels of specific species of mRNA. One method for doing this is known as TAQMAN™ (PE Applied Biosystems, Foster City, Calif., USA), described in greater detail infra.

Other ways of measuring the transcriptional state of a cell include producing pools of restriction fragments of limited complexity for electrophoretic analysis, such as by methods combining double restriction enzyme digestion with phasing primers (see, e.g., EP 0 534858 A1, filed Sep. 24, 1992, by Zabeau et al.), or methods selecting restriction fragments with sites closest to a defined mRNA end. See, e.g., Prashar & Weissman, Proc. Natl. Acad. Sci. USA 93(2) 659-663 (1996). Still other methods including generating statistically sample cDNA pools, such as by sequencing sufficient bases, e.g., 20-50 bases, in each of multiple cDNAs to identify each cDNA, and sequencing short tags, e.g., 9-10 bases, which are generated at known positions relative to a defined mRNA end pathway pattern. See, e.g., Velculescu, Science 270: 484-487 (1995).

Standard control levels of a gene expression product are determined by measuring gene expression in different control groups. The control group gene expression levels are then compared with the measured level of a gene expression product in a given subject. This gene expression product could be the characteristic mRNA associated with that particular genotype group or the polypeptide gene expression product of that genotype group. The patient could then be classified or assigned to a particular genotype group based on how similar the measured levels were compared to the control levels for a given group.

As one of skill in the art will understand, there will be a certain degree of uncertainty involved in making this determination. Therefore, the standard deviations of the control group levels would be used to make a probabilistic determination and the methods of this invention would be applicable over a wide range of probability based genotype group determinations. Thus, for example and not by way of limitation, in one embodiment, if the measured level of the gene expression product falls within 2.5 standard deviations of the mean of any of the control groups, then that individual may be assigned to that genotype group. In another embodiment if the measured level of the gene expression product falls within 2.0 standard deviations of the mean of any of the control groups then that individual may be assigned to that genotype group. In still another embodiment, if the measured level of the gene expression product falls within 1.5 standard deviations of the mean of any of the control groups then that individual may be assigned to that genotype group. In yet another embodiment, if the measured level of the gene expression product is 1.0 or less standard deviations of the mean of any of the control groups levels then that individual may be assigned to that genotype group.

Thus this process will allow the determining, with various degrees of probability, which group a specific patient should be placed in, and such assignment to a genotype group would then determine the risk category into which the individual should be placed.

Methods to detect and measure mRNA levels and levels of polypeptide gene expression products are well known in the art and include the use of nucleotide microarrays and polypeptide detection methods involving mass spectrometers and/or antibody detection and quantification techniques. See also, Tom Strachan & Andrew Read, Human Molecular Genetics, 2^{nd} Edition. (John Wiley and Sons, Inc. Publication, NY, 1999).

Measurement of Other Aspects. In various embodiments of the present invention, aspects of the biological state other than the transcriptional state, such as the translational state, the activity state, or mixed aspects can be measured in order to obtain drug and pathway responses. Details of these embodiments are described below.

Translational state measurements. Expression of the protein encoded by the genes of the invention can be detected by a probe which is detectably-labelled, or which can be subsequently-labelled. Generally, the probe is an antibody that recognizes the expressed protein.

The term “antibody” includes, but is not limited to, polyclonal antibodies, monoclonal antibodies, humanized or chimeric antibodies and biologically functional antibody fragments sufficient for binding of the antibody fragment to the protein.

For the production of antibodies to a protein encoded by one of the disclosed genes, various host animals may be immunized by injection with the polypeptide, or a portion thereof. Such host animals may include, but are not limited to, rabbits, mice and rats. Various adjuvants may be used to increase the immunological response, depending on the host species including, but not limited to, Freund's (complete and incomplete), mineral gels, such as aluminum hydroxide; surface active substances, such as lysolecithin, pluronic polyols, polyanions, peptides, oil emulsions, keyhole limpet hemocyanin and dinitrophenol; and potentially useful human adjuvants, such as bacille Camette-Guerin (BCG) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler & Milstein, Nature 256: 495-497 (1975); and U.S. Pat. No. 4,376,110; the human B-cell hybridoma technique of Kosbor et al., Immunol. Today 4: 72 (1983); Cole et al., Proc. Natl. Acad. Sci. USA 80: 2026-2030 (1983); and the EBV-hybridoma technique, Cole et al., Monoclonal Antibodies and Cancer Therapy pp. 77-96 (Alan R. Liss, Inc., 1985). Such antibodies may be of any immunoglobulin class including IgG, IgM, IgE, IgA, IgD and any subclass thereof. The hybridoma producing the mAb of this invention may be cultivated in vitro or in vivo. Production of high titres of mAbs in vivo makes this the presently preferred method of production.

In addition, techniques developed for the production of “chimeric antibodies” (see Morrison et al., Proc. Natl. Acad. Sci. USA 81: 6851-6855 (1984); Neuberger et al., Nature 312: 604-608 (1984); and Takeda et al., Nature 314: 452-454 (1985)), by splicing the genes from a mouse antibody molecule of appropriate antigen specificity together with genes from a human antibody molecule of appropriate biological activity, can be used. A chimeric antibody is a molecule in which different portions are derived from different animal species, such as those having a variable or hypervariable region derived form a murine mAb and a human immunoglobulin constant region.

Alternatively, techniques described for the production of single chain antibodies, U.S. Pat. No. 4,946,778; Bird, Science 242: 423-426 (1988); Huston et al., Proc. Natl. Acad. Sci. USA 85: 5879-5883 (1988); and Ward et al., Nature 334: 544-546 (1989), can be adapted to produce differentially expressed gene single-chain antibodies. Single-chain antibodies are formed by linking the heavy- and light-chain fragments of the Fv region via an amino acid bridge, resulting in a single-chain polypeptide.

Techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments, which recognize specific epitopes, may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (see Huse et al., Science 246: 1275-1281 (1989)), to allow rapid and easy identification of monoclonal Fab fragments with the desired specificity.

The extent to which the known proteins are expressed in a given sample is then determined by immunoassay methods that utilize the antibodies described above. Such immunoassay methods include, but are not limited to, dot blotting, western blotting, competitive and non-competitive protein binding assays, enzyme-linked immunosorbant assays (ELISA), immunohistochemistry, fluorescence activated cell sorting (FACS), and others commonly used and widely-described in scientific and patent literature, and many employed commercially.

Particularly preferred, for ease of detection, is the sandwich ELISA, of which a number of variations exist, all of which are intended to be used in the methods and assays of the present invention. For example, in a typical forward assay, unlabeled antibody is immobilized on a solid substrate and the sample to be tested brought into contact with the bound molecule after a suitable period of incubation, for a period of time sufficient to allow formation of an antibody-antigen binary complex. At this point, a second antibody, labelled with a reporter molecule capable of inducing a detectable signal, is then added and incubated, allowing time sufficient for the formation of a ternary complex of antibody-antigen-labelled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labelled antibody and sample to be tested are first combined, incubated and added to the unlabeled surface bound antibody. These techniques are well-known to those skilled in the art, and the possibility of minor variations will be readily apparent. As used herein, “sandwich assay” is intended to encompass all variations on the basic two-site technique. For the immunoassays of the present invention, the only limiting factor is that the labelled antibody must be an antibody that is specific for the protein expressed by the gene of interest.

The most commonly used reporter molecules in this type of assay are either enzymes, fluorophore- or radionuclide-containing molecules. In the case of an enzyme immunoassay (EIA) an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, β-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of protein which is present in the serum sample.

Alternately, fluorescent compounds, such as fluorescein and rhodamine, may be chemically coupled to antibodies without altering their binding capacity. When activated by illumination with light of a particular wavelength, the fluorochrome-labelled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well-established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Measurement of the translational state of the genes of the invention may also be performed according to several additional methods. For example, whole genome monitoring of protein, i.e., the “proteome,” Goffeau et al., supra, can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. Methods for making monoclonal antibodies are well-known. See, e.g., Harlow & Lane, Antibodies: A Laboratory Manual (Cold Spring Harbor, N.Y., 1988), which is incorporated in its entirety for all purposes. In a preferred embodiment, monoclonal antibodies are raised against synthetic peptide fragments designed based on genomic sequence of the cell. With such an antibody array, proteins from the cell are contacted to the array, and their binding is assayed with assays known in the art.

Proteins can be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., Gel Electrophoresis of Proteins: A Practical Approach (IRL Press, NY, 1990); Shevchenko et al., Proc. Natl. Acad. Sci. USA 93: 14440-14445 (1996); Sagliocco et al., Yeast 12: 1519-1533 (1996); and Lander, Science 274: 536-539 (1996). The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells, e.g., in yeast, exposed to a drug, or in cells modified by, e.g., deletion or over-expression of a specific gene.

Detection of the polypeptide (protein) expression product of the gene in body fluids or tissues can be used to determine the presence or absence of the polymorphism, and the relative level of the polypeptide expression product can be used to determine if the polymorphism is present in a homozygous or heterozygous state (and hence the risk category of the individual).

Embodiments Based on Other Aspects of the Biological State. Although monitoring cellular constituents other than mRNA abundances currently presents certain technical difficulties, it will be apparent to those of skill in the art employing the methods of this invention that the activities of proteins relevant to the characterization of cell function can be measured, and embodiments of this invention can be based on such measurements. Activity measurements can be performed by any functional, biochemical or physical means appropriate to the particular activity being characterized. Where the activity involves a chemical transformation, the cellular protein can be contacted with natural substrates, and the rate of transformation measured. Where the activity involves association in multimeric units, e.g., association of an activated DNA binding complex with DNA, the amount of associated protein or secondary consequences of the association, such as amounts of mRNA transcribed, can be measured. Also, where only a functional activity is known, e.g., as in cell cycle control, performance of the function can be observed. However known and measured, the changes in protein activities form the response data analyzed by the methods of this invention.

In alternative and non-limiting embodiments, response data may be formed of mixed aspects of the biological state of a cell. Response data can be constructed from, e.g., changes in certain mRNA abundances, changes in certain protein abundances and changes in certain protein activities.

The Detection of Nucleic Acids and Proteins as Markers. In a particular embodiment, the level of mRNA corresponding to a marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells. See, e.g., Ausubel et al., Ed., Curr. Prot. Mol. Biol. (John Wiley & Sons, NY, 1987-1999). Additionally, large numbers of tissue samples can readily be processed using techniques well-known to those of skill in the art, such as, e.g., the single-step RNA isolation process of U.S. Pat. No. 4,843,155.

The isolated mRNA can be used in hybridization or amplification assays that include, but are not limited to, Southern or Northern analyses, PCR analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, e.g., a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example, by running the isolated mRNA on an agarose gel and transferring the mRNA from the gel to a membrane, such as nitrocellulose. In an alternative format, the probes are immobilized on a solid surface and the mRNA is contacted with the probes, for example, in an Affymetrix gene chip array. A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by RT-PCR (the experimental embodiment set forth in Mullis, U.S. Pat. No. 4,683,202 (1987); ligase chain reaction, Barany (1991), supra; self-sustained sequence replication, Guatelli et al., Proc. Natl. Acad. Sci. USA 87: 1874-1878 (1990); transcriptional amplification system, Kwoh et al., Proc. Natl. Acad. Sci. USA 86: 1173-1177 (1989); Q-Beta Replicase, Lizardi et al., Biol. Technology 6: 1197 (1988); rolling circle replication, U.S. Pat. No. 5,854,033 (1988); or any other nucleic acid amplification method, followed by the detection of the amplified molecules using techniques well-known to those of skill in the art. These detection schemes are especially useful for the detection of the nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10-30 nucleotides in length and flank a region from about 50-200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes, such as the actin gene or epithelial cell-specific genes. This normalization allows the comparison of the expression level in one sample, e.g., a patient sample, to another sample or between samples from different sources.

Alternatively, the expression level can be provided as a relative expression level. To determine a relative expression level of a marker, the level of expression of the marker is determined for 10 or more samples of normal versus disease biological samples, preferably 50 or more samples, prior to the determination of the expression level for the sample in question. The mean expression level of each of the genes assayed in the larger number of samples is determined and used as a baseline expression level for the marker. The expression level of the marker determined for the test sample (absolute level of expression) is then divided by the mean expression value obtained for that marker. This provides a relative expression level.

Preferably, the samples used in the baseline determination will be from subjects who do not have the polymorphism. The choice of the cell source is dependent on the use of the relative expression level. Using expression found in normal tissues as a mean expression score aids in validating whether the marker assayed is specific (versus normal cells). In addition, as more data is accumulated, the mean expression value can be revised, providing improved relative expression values based on accumulated data.

Detection of Polypeptides. In another embodiment of the present invention, a polypeptide corresponding to a marker is detected. A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof, e.g., Fab or F(ab′)₂ can be used. The term “labelled,” with regard to the probe or antibody, is intended to encompass direct-labeling of the probe or antibody by coupling, i.e., physically linking, a detectable substance to the probe or antibody, as well as indirect-labelling of the probe or antibody by reactivity with another reagent that is directly-labelled. Examples of indirect labelling include detection of a primary antibody using a fluorescently-labelled secondary antibody and end-labelling of a DNA probe with biotin such that it can be detected with fluorescently-labelled streptavidin.

Proteins from individuals can be isolated using techniques that are well-known to those of skill in the art. The protein isolation methods employed can, e.g., be such as those described in Harlow & Lane (1988), supra.

A variety of formats can be employed to determine whether a sample contains a protein that binds to a given antibody. Examples of such formats include, but are not limited to, EIA, radioimmunoasay (RIA), Western blot analysis and ELISA. A skilled artisan can readily adapt known protein/antibody detection methods for use in determining whether cells express a marker of the present invention and the relative concentration of that specific polypeptide expression product in blood or other body tissues.

In one format, antibodies or antibody fragments can be used in methods, such as Western blots or immunofluorescence techniques, to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros and magnetite.

One skilled in the art will know many other suitable carriers for binding antibody or antigen, and will be able to adapt such support for use with the present invention. For example, protein isolated from patient cells can be run on a polyacrylamide gel electrophoresis and immobilized onto a solid phase support, such as nitrocellulose. The support can then be washed with suitable buffers followed by treatment with the detectably-labelled antibody. The solid phase support can then be washed with the buffer a second time to remove unbound antibody. The amount of bound label on the solid support can then be detected by conventional means and this measurement translated into a level or concentration of protein in blood or another body tissue.

Kits. The invention also encompasses kits for detecting the presence of a polypeptide or nucleic acid corresponding to a marker of the invention in a biological sample, e.g., any bodily fluid including, but not limited to, serum, plasma, lymph, cystic fluid, urine, stool, cerebrospinal fluid, acitic fluid or blood, and including biopsy samples of body tissue. For example, the kit can comprise a labelled compound or agent capable of detecting a polypeptide or an mRNA encoding a polypeptide corresponding to a marker of the invention in a biological sample and means for determining the amount of the polypeptide or mRNA in the sample, e.g., an antibody which binds the polypeptide or an oligonucleotide probe which binds to DNA or mRNA encoding the polypeptide. Kits can also include instructions for interpreting the results obtained using the kit.

In another embodiment, the invention provides a kit comprising at least two genotyping oligonucleotides packaged in separate containers. The kit may also contain other components such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as in the case of PCR.

In a preferred embodiment, such kit may further comprise a DNA sample collecting means.

In particular, the genotyping primer composition may comprise at least two sets of allele specific primer pairs. Preferably, the two genotyping oligonucleotides are packaged in separate containers.

For antibody-based kits, the kit can comprise, e.g., 1) a first antibody, e.g., attached to a solid support, which binds to a polypeptide corresponding to a marker or the invention; and, optionally; 2) a second, different antibody which binds to either the polypeptide or the first antibody and is conjugated to a detectable label.

For oligonucleotide-based kits, the kit can comprise, e.g., 1) an oligonucleotide, e.g., a detectably-labelled oligonucleotide, which hybridizes to a nucleic acid sequence encoding a polypeptide corresponding to a marker of the invention; or 2) a pair of primers useful for amplifying a nucleic acid molecule corresponding to a marker of the invention.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit.

The following EXAMPLES are presented to more fully illustrate the preferred embodiments of the invention. These EXAMPLES should in no way be construed as limiting the scope of the invention, as defined by the appended claims.

### Example I

### Pharmacogenetic Analysis of Response to Tegaserod Patients with Chronic Constipation

Introduction. A clinical trial was performed to evaluate the efficacy of tegaserod (Zelmac®/Zelnorm™) at 6 mg bid (12 mg/day) or 2 mg bid (4 mg/day) vs. placebo by measuring the number of complete spontaneous bowel movements (CSBM) during the first 4 weeks of treatment. Secondary objectives included evaluating the number of CSBM during the entire 12-week treatment period, bowel habits, laxative use, and safety and tolerability issues. The clinical trial design consisted of a 2-week baseline period without medication, a 12-week, randomized, double-blind, placebo-controlled treatment period, and a 4-week withdrawal period without study medication. As part of the clinical trial, DNA was collected from patients at screening for pharmacogenetic analysis to evaluate genetic polymorphisms relating to drug targets and disease aetiology.

Two major classes of insertion/deletion polymorphisms have been characterized for the SLC6A4 gene, one in intron 2 and another in the promoter region. SLC6A4 is the serotonin transporter gene, also know as SERT or HTT. Camilleri and colleagues reported that SLC6A4 promoter insertion/deletion polymorphism was associated with colonic transit response following treatment with the serotonin 5-HT₃ receptor antagonist alosetron. Camilleri M et al., Gastroenterology; 123 (2):425-32 (2002). We found that there was a trend for individuals homozygous for the C16 (long) allele of the serotonin transporter promoter polymorphism to display greater response to tegaserod compared to placebo. The overall response rates for C16-C16 were 48% for tegaserod and 21% for placebo (OR=3.51, 95% CI: 1.67-7.41) compared to the C14-C14 homozygotes at 45% for tegaserod and 27% for placebo (OR=2.3, 95% CI: 0.93-5.69) and C14-C16 heterozygotes at 43% for tegaserod and 28% for placebo (OR=1.95; 95% CI: 1.12-3.41). Within a given treatment group, there was no statistically significant difference between response rates between the different genotypes.

In this study, a total of 55 single nucleotide polymorphisms (SNPs) in 23 new candidate genes were evaluated (see TABLE 1 for complete list). These included a number of SNPs in the drug target (HTR4) as well as in 5-HT₃ receptor subunits HTR3A and HTR3B. Genes related to serotonin synthesis (TPH, TDO2) and downstream serotonin signalling (CALCA or CGRP1), as well as genes related to gut motility such as motilin (MLN) and gut secretion (SLC12A2, AQP3, SCNNLA) were also evaluated.

TABLE 1
Candidate Gene List

| Gene | Description | Function/Comments |
|---|---|---|
| ABP1 | amiloride binding protein 1 | inhibition by pentylaminoguanidine |
| | (amine oxidase (copper-containing)); | (PAG), a degradation product of |
| | diamine oxidase (DAO) | tegaserod; chosen based on possible |
| | | safety concern of mucosal hyperplasia |
| | | seen in mouse |
| ADORA1 | adenosine A1 receptor | G protein-coupled receptor that |
| | | selectively binds adenosine; involved |
| | | in enteric nervous system |
| AQP3 | aquaporin 3 | member of the aquaporin family of |
| | | intrinsic membrane proteins that |
| | | function as water-selective channels |
| | | in the plasma membranes of many cells |
| AQP4 | aquaporin 4 | water channel transporting water, |
| | | glycerol, or other small molecules; |
| | | member of the MIP family of proteins |
| CALCA | calcitonin/calcitonin-related | Precursor of calcitonin and calcitonin |
| | polypeptide, alpha (CGRP1) | gene-related peptide; ligands for |
| | | receptors found in nervous system |
| | | and peripheral tissues |
| CFTR | cystic fibrosis transmembrane | Cl transport; regulator of ENaC |
| | regulator | sodium channels |
| HTR3A | 5-hydroxytryptamine (serotonin) | activation by serotonin involved |
| | receptor 3A | in initiating peristaltic reflex |
| | | and facilitating intraluminal |
| | | secretions |
| HTR3B | 5-hydroxytryptamine (serotonin) | activation by serotonin involved in |
| | receptor 3B | initiating peristaltic reflex and |
| | | facilitating intraluminal secretions |
| HTR4 | 5-hydroxytryptamine (serotonin) | Target of Zelmac; activation by |
| | receptor 4 | serotonin involved in initiating |
| | | peristaltic reflex and facilitating |
| | | intraluminal secretions |
| MLN | motilin | stimulates contractions of the |
| | | antrum and duodenum |
| SCNN1A | sodium channel, non voltage-gated 1 | Na transport |
| | alpha | |
| SGK | serum &glucocorticoid regulatory | important regulatory protein in |
| | kinase | the colon, mediates early |
| | | aldosterone action |
| SLC12A2 | solute carrier family 12 | NKCC1 - solute tranpsort |
| | (sodium/potassium/chloride | |
| | transporters), member 2 | |
| SLC12A4 | solute carrier family 12 | KCC1 solute transport |
| | (potassium/chloride transporters), | |
| | member 4 | |
| SLC26A3 | solute carrier family 26, member 3 | Tumor suppressor expressed in colon; |
| | | has probable anion transporter |
| | | activity |
| SLC4A1 | solute carrier family 4, anion | AE1 - anion exchange 1 |
| | exchanger, member 1 (erythrocyte | |
| | membrane protein band 3, Diego | |
| | blood group) | |
| SLC4A2 | solute carrier family 4, anion | AE2 - anion exchange 2 |
| | exchanger, member 2 (erythrocyte | |
| | membrane protein band 3-like 1) | |
| SLC9A2 | solute carrier family 9 | electroneutrol NaCl transport |
| | (sodium/hydrogen exchanger), isoform | |
| | 2 (NHE2) | |
| SLC9A3 | solute carrier family 9 | electroneutrol NaCl transport |
| | (sodium/hydrogen exchanger), isoform | |
| | 3 (NHE3) | |
| SLC9A3R1 | solute carrier family 9 | Regulatory cofactor of the |
| | (sodium/hydrogen exchanger), | NHE3 (SLC9A3) sodium/hydrogen |
| | isoform 3 regulatory factor 1 | antiporter |
| TAC1 | tachykinin, precursor 1 (substance K, | hormones thought to function |
| | substance P, neurokinin 1, neurokinin 2, | as neurotransmitters which |
| | neuromedin L, neurokinin alpha, | interact with nerve receptors |
| | neuropeptide K, neuropeptide gamma) | and smooth muscle cells; |
| | | function as vasodilators |
| | | and secretagogues |
| TDO2 | tryptophan 2,3-dioxygenase | catalyzes rate-limiting |
| | | step in catabolism of |
| | | tryptophan to serotonin |
| TPH | tryptophan hydroxylase | catalyzes the initial and |
| | (tryptophan 5-monooxygenase) | rate limiting step in the |
| | | synthesis of serotonin |

Samples. A total 1348 patients were randomized as the intent-to-treat (ITT) population. Blood samples from these patients were collected at the time of patient screening and DNA was extracted using the PUREGENE™ DNA Isolation Kit (D-50K). Of these, samples from 738 randomized patients had good quality genotype data for at least half of the 55 SNPs evaluated.

Clinical assessments. Primary efficacy was measured by the number of complete spontaneous bowel movements (CSBM), where “complete” refers to a bowel movement that results in a feeling of complete evacuation and “spontaneous” indicates that no laxatives were used within 24 hours. The primary efficacy endpoint evaluated was the increase of 1 or more CSBM/week over baseline after 4 weeks of treatment. A secondary endpoint evaluated in this genotyping study was RESP12, the increase of 1 of more CSBM/week over baseline after 12 weeks of treatment. Patients were classified as responders if they met these criteria and had completed at least 7 days of treatment. Additional secondary efficacy assessments were evaluated based on the weekly diary assessments, such as bothersome constipation, bothersome abdominal pain, and satisfaction with bowel habits at the end of the 12-week treatment period (using the baseline value as a co-variant for analysis).

Genotyping. A total of 61 polymorphisms in 23 candidate genes were selected for analysis. Genotyping was performed using the TaqMan® technology for most of the assays, while a few were performed in-house using the Third Wave Technologies Invader Assay technique (see TABLE 2 for assay IDs for all evaluated SNPs). Of the 61 SNPs genotyped, 55 yielded good quality, polymorphic genotypes for use in further evaluation.

TABLE 2
Assay IDs for Evaluated Candidate Genes

| GENE | PG ID | ASSAY NAME |
|---|---|---|
| ABP1 | 1763 | TWT_402234 |
| ABP1 | 1764 | ABI_E_rs2071514_10 |
| ABP1 | 2185 | ABI_E_rs1049793_10 |
| ADORA1 | 1786 | ABI_C____262337_1_{—} |
| AQP3 | 1838 | ABI_E__rs517210_10 |
| AQP3 | 1839 | ABI_C___2736449_1_{—} |
| AQP3 | 1840 | TWT_402221 |
| AQP4 | 1841 | ABI_E_rs2339214_10 |
| CALCA | 3750 | ABI_C___2697070_1_{—} |
| CALCA | 3783 | ABI_E____rs5241_10 |
| CFTR | 3745 | ABI_C____656774_1_{—} |
| CFTR | 3752 | ABI_C___3021357_10 |
| CFTR | 3761 | ABI_C____656763 10 |
| HTR3A | 1748 | ABI_C___1372135_1_{—} |
| HTR3A | 1749 | ABI_E_rs1176713_10 |
| HTR3B | 3755 | ABI_C___7488596_1_{—} |
| HTR4 | 1746 | ABI_E__rs723180_10 |
| HTR4 | 3741 | ABI_C__11259587_10 |
| HTR4 | 3742 | ABI_C__11259592_10 |
| HTR4 | 3743 | ABI_C__11267705_10 |
| HTR4 | 3747 | ABI_C___1992167_10 |
| HTR4 | 3751 | ABI_C___2816608_10 |
| HTR4 | 3753 | ABI_C___3168086 10 |
| HTR4 | 3754 | ABI_C___3220820_10 |
| HTR4 | 3756 | ABI_C___7505278_10 |
| HTR4 | 3760 | ABI_C____349748_10 |
| MLN | 1783 | ABI_C___1920380_1_{—} |
| MLN | 3748 | ABI_C___2005027_10 |
| SCNN1A | 1804 | ABI_E_rs2228576_10 |
| SCNN1A | 3805 | ABI_E_rs3741914_10 |
| SCNN1A | 3806 | ABI_E_rs3782726_10 |
| SGK | 3744 | ABI_C___1347310_10 |
| SLC12A2 | 1842 | ABI_C___8942125_1_{—} |
| SLC12A2 | 1843 | ABI_E__rs790156_10 |
| SLC12A2 | 3801 | ABI_E_rs1864922_10 |
| SLC12A4 | 1829 | ABI_E_rs3785098_10 |
| SLC26A3 | 1835 | ABI_C___8856481_10 |
| SLC26A3 | 1837 | ABI_E_rs2269778_10 |
| SLC26A3 | 3804 | ABI_E_rs3735605_10 |
| SLC4A1 | 1822 | ABI_C___2548337_1_{—} |
| SLC4A1 | 1824 | ABI_E_rs2072081_10 |
| SLC4A1 | 3782 | ABI_E____rs5036_10 |
| SLC4A2 | 1825 | TWT_402225 |
| SLC4A2 | 3342 | ABI_C__15972956_10 |
| SLC4A2 | 3344 | ABI_C___2073105_1_{—} |
| SLC9A2 | 1828 | TWT_402224 |
| SLC9A2 | 3758 | ABI_C___8906170_10 |
| SLC9A2 | 3759 | ABI_C___8906179_10 |
| SLC9A3 | 3757 | ABI_C___8293157_10 |
| SLC9A3R1 | 1819 | ABI_C___2160161_1_{—} |
| SLC9A3R1 | 3749 | ABI_C___2160155_10 |
| TAC1 | 1795 | ABI_C___2560484_1_{—} |
| TDO2 | 3746 | ABI_C___1601897_10 |
| TPH | 1756 | ABI_C___2298450_1_{—} |
| TPH | 3784 | ABI_E_rs1607395_10 |

ABI = Applied Biosystems Inc.; TWT = Third Wave Technologies

Statistical analysis. Statistical analysis was performed using SAS version 8.2 (The SAS Institute, Cary, N.C., USA). Analysis methods included Fisher's Exact tests, analysis of Covariance (ANCOVA), logistic regression, and Mantel-Haenzel tests to determine odds ratios and 95% confidence intervals, with significance established at p<0.05. Calculation of linkage disequilibrium was performed using the GOLD (graphical overview of linkage disequilibrium) method described by Abecasis & Cookson, Bioinformatics 16: 182-3 (2000).

Demographic comparisons. TABLE 3 presents the demographic breakdown of the patients in the overall clinical study and those included in this genotype analysis. Distributions were similar between the genotype population and the total patient population. A comparison of genotype by race demonstrated that 39 of the 55 SNPs had significant differences in genotype distribution by race (p<0.05 Fishers Exact test). Based on these findings, it was decided to limit analysis to the Caucasian population, which accounted for nearly 90% of all patients, to prevent a confounding effect of race on subsequent genotype analyses. Furthermore, the analysis was also limited to those intent-to-treat patients that were treated with tegaserod or placebo for at least 7 days (total of 635 patients).

TABLE 3
Demographics of Patients Genotyped

| All ITT patients | | | | ITT Caucasians, >=7 days of Trt | | | |
|---|---|---|---|---|---|---|---|
| All Patients | | Genotyped | | All Patients | | Genotyped | |
| N | (%) | N | (%) | N | (%) | N | (%) |

Gender

| | | | | | | | | |
|---|---|---|---|---|---|---|---|---|
| Male (SEX1C = 1) | 135 | (10.0) | 86 | (11.7) | 113 | (10.1) | 70 | (11.0) |
| Female (SEX1C = 2) | 1213 | (90.0) | 652 | (88.4) | 1006 | (89.9) | 565 | (89.0) |

Race

| | | | | | | | | |
|---|---|---|---|---|---|---|---|---|
| Caucasian (RCE1C = 1) | 1142 | (84.7) | 646 | (87.5) | 1119 | (100.0) | 635 | (100.0) |
| Black (RCE1C = 2) | 96 | (7.1) | 47 | (6.4) | — | — | — | — |
| Oriental (RCE1C = 3) | 6 | (0.5) | 5 | (0.7) | — | — | — | — |
| Other (RCE1C = 4) | 104 | (7.7) | 40 | (5.4) | — | — | — | — |

Age Category

| | | | | | | | | |
|---|---|---|---|---|---|---|---|---|
| <65 (AGECAT = 1) | 1188 | (88.1) | 648 | (87.8) | 971 | (86.8) | 548 | (86.3) |
| > = 65 (AGECAT = 2) | 160 | (11.9) | 90 | (12.2) | 148 | (13.2) | 87 | (13.7) |

Treatment

| | | | | | | | | |
|---|---|---|---|---|---|---|---|---|
| Tegaserod 6-mg bid (TRTC = B2) | 451 | (33.5) | 240 | (32.5) | 375 | (33.5) | 204 | (32.1) |
| Tegaserod 2-mg bid (TRTC = C2) | 450 | (33.4) | 260 | (35.2) | 375 | (33.5) | 227 | (35.8) |
| Placebo (TRTC = P) | 447 | (33.2) | 238 | (32.3) | 369 | (33.0) | 204 | (32.1) |
| Total | 1348 | | 738 | | 1119 | | 635 | |

ITT = intent to treat; N = number of patients; Trt = treatment

Comparison of overall response rates. TABLE 4 presents the overall response for the primary efficacy endpoint (a mean increase in 1 or more CSBM/week over baseline after 4 weeks) for tegaserod 6-mg bid compared with placebo for both the overall patient population and the subgroup of patients for which we had genotype data (all races and Caucasians only). In all cases, individuals treated with tegaserod reported higher rates of response compared to those treated with placebo, although a statistically significant difference was not seen in the males or in the elderly population. This was also seen with the lower dose (2-mg bid) of tegaserod, although the response was less than that seen for the 6-mg bid dose. Further analysis was limited to comparisons between only the 6-mg bid tegaserod-treated and the placebo-treated patients.

TABLE 4
Response rates for tegaserod 6-mg bid vs. placebo after 4 weeks of
treatment: comparison between all patients and genotyped patients

| | | Number of Patients | | | % Responders | | Odds | | |
|---|---|---|---|---|---|---|---|---|---|
| Age | Gender | Teg-6 | Plac | Total | Teg-6 | Plac | Ratio | (95% CI) | p-value |

All patients (ITT, at least 7 days of treatment)

| | | | | | | | | | |
|---|---|---|---|---|---|---|---|---|---|
| All | M + F | 439 | 438 | 877 | 44% | 25% | 2.3 | (1.8, 3.1) | <0.0001 |
| All | Female | 397 | 398 | 795 | 45% | 26% | 2.4 | (1.8, 3.2) | <0.0001 |
| All | Male | 42 | 40 | 82 | 36% | 23% | 1.9 | (0.7, 5.1) | 0.1914 |
| <65 | M + F | 399 | 379 | 778 | 45% | 25% | 2.5 | (1.8, 3.3) | <0.0001 |
| >=65 | M + F | 40 | 59 | 99 | 35% | 27% | 1.4 | (0.6, 3.4) | 0.4048 |

All Caucasians (ITT, at least 7 days of treatment)

| | | | | | | | | | |
|---|---|---|---|---|---|---|---|---|---|
| All | M + F | 375 | 369 | 744 | 45% | 25% | 2.5 | (1.8, 3.4) | <0.0001 |
| All | Female | 337 | 332 | 669 | 46% | 25% | 2.6 | (1.8, 3.5) | <0.0001 |
| All | Male | 38 | 37 | 75 | 37% | 24% | 1.8 | (0.7, 4.9) | 0.2430 |
| <65 | M + F | 336 | 315 | 651 | 46% | 24% | 2.7 | (1.9, 3.8) | <0.0001 |
| >=65 | M + F | 39 | 54 | 93 | 33% | 30% | 1.2 | (0.5, 2.9) | 0.7051 |

All genotyped (ITT, at least 7 days of treatment)

| | | | | | | | | | |
|---|---|---|---|---|---|---|---|---|---|
| All | M + F | 234 | 234 | 468 | 46% | 24% | 2.7 | (1.8, 4.0) | <0.0001 |
| All | Female | 209 | 208 | 417 | 45% | 24% | 2.7 | (1.8, 4.1) | <0.0001 |
| All | Male | 25 | 26 | 51 | 48% | 27% | 2.5 | (0.8, 8.1) | 0.1233 |
| <65 | M + F | 216 | 203 | 419 | 45% | 23% | 2.8 | (1.8, 4.2) | <0.0001 |
| >=65 | M + F | 18 | 31 | 49 | 50% | 29% | 2.4 | (0.7, 8.2) | 0.1463 |

Caucasians genotyped (ITT, at least 7 days of treatment)

| | | | | | | | | | |
|---|---|---|---|---|---|---|---|---|---|
| All | M + F | 204 | 204 | 408 | 46% | 25% | 2.6 | (1.7, 4.0) | <0.0001 |
| All | Female | 183 | 181 | 364 | 45% | 24% | 2.7 | (1.7, 4.2) | <0.0001 |
| All | Male | 21 | 23 | 44 | 52% | 30% | 2.5 | (0.7, 8.6) | 0.1437 |
| <65 | M + F | 187 | 174 | 361 | 46% | 24% | 2.8 | (1.8, 4.3) | <0.0001 |
| >=65 | M + F | 17 | 30 | 47 | 47% | 30% | 2.1 | (0.6, 7.1) | 0.2473 |

Teg-6 = tegaserod 6-mg bid; Plac = placebo; CI = confidence interval; ITT = intent to treat; M + F = males and females

The overall response rate to tegaserod 6-mg bid was 46% compared to 25% in placebo for our genotyped population (Caucasians with at least 7 days of treatment). The calculated odds ratio, which indicates the likelihood of achieving a positive response given treatment as compared to placebo, was equal to 2.6 (95% CI: 1.7-4.0). These values were highly statistically significant and were comparable to values calculated for the clinical trial as a whole (see TABLE 4). Response rates were similar between the patient populations with the exception of the response in males and the elderly, which appeared to be somewhat higher in the genotyped subpopulation. Similar results were seen following 12 weeks are treatment, as presented in TABLE 5.

TABLE 5
Response rates for tegaserod 6-mg bid vs. placebo after 12 weeks of
treatment; comparison between all patients and genotyped patients

| | | Number of Patients | | | % Responders | | Odds | | |
|---|---|---|---|---|---|---|---|---|---|
| Age | Gender | Teg-6 | Plac | Total | Teg-6 | Plac | Ratio | (95% CI) | p-value |

All patients (ITT, at least 7 days of treatment)

| | | | | | | | | | |
|---|---|---|---|---|---|---|---|---|---|
| All | M + F | 439 | 438 | 877 | 46% | 27% | 2.3 | (1.7, 3.0) | <0.0001 |
| All | Female | 397 | 398 | 795 | 46% | 28% | 2.2 | (1.7, 3.0) | <0.0001 |
| All | Male | 42 | 40 | 82 | 43% | 23% | 2.6 | (1.0, 6.8) | 0.0513 |
| <65 | M + F | 399 | 379 | 778 | 46% | 27% | 2.3 | (1.7, 3.2) | <0.0001 |
| >=65 | M + F | 40 | 59 | 99 | 40% | 29% | 1.6 | (0.7, 3.8) | 0.2490 |

All Caucasians (ITT, at least 7 days of treatment)

| | | | | | | | | | |
|---|---|---|---|---|---|---|---|---|---|
| All | M + F | 375 | 369 | 744 | 47% | 26% | 2.5 | (1.9, 3.4) | <0.0001 |
| All | Female | 337 | 332 | 669 | 47% | 26% | 2.5 | (1.8, 3.5) | <0.0001 |
| All | Male | 38 | 37 | 75 | 45% | 24% | 2.5 | (0.9, 6.8) | 0.0651 |
| <65 | M + F | 336 | 315 | 651 | 48% | 25% | 2.8 | (2.0, 3.9) | <0.0001 |
| >=65 | M + F | 39 | 54 | 93 | 38% | 31% | 1.4 | (0.6, 3.2) | 0.4868 |

All genotyped (ITT, at least 7 days of treatment)

| | | | | | | | | | |
|---|---|---|---|---|---|---|---|---|---|
| All | M + F | 234 | 234 | 468 | 47% | 26% | 2.6 | (1.7, 3.8) | <0.0001 |
| All | Female | 209 | 208 | 417 | 47% | 26% | 2.5 | (1.7, 3.8) | <0.0001 |
| All | Male | 25 | 26 | 51 | 52% | 27% | 2.9 | (0.9, 9.5) | 0.0694 |
| <65 | M + F | 216 | 203 | 419 | 47% | 25% | 2.6 | (1.7, 4.0) | <0.0001 |
| >=65 | M + F | 18 | 31 | 49 | 56% | 32% | 2.6 | (0.8, 8.7) | 0.1134 |

Caucasians genotyped (ITT, at least 7 days of treatment)

| | | | | | | | | | |
|---|---|---|---|---|---|---|---|---|---|
| All | M + F | 204 | 204 | 408 | 49% | 25% | 2.8 | (1.9, 4.3) | <0.0001 |
| All | Female | 183 | 181 | 364 | 48% | 25% | 2.8 | (1.8, 4.4) | <0.0001 |
| All | Male | 21 | 23 | 44 | 57% | 30% | 3.0 | (0.9, 10.5) | 0.0774 |
| <65 | M + F | 187 | 174 | 361 | 49% | 24% | 3.0 | (1.9, 4.7) | <0.0001 |
| >=65 | M + F | 17 | 30 | 47 | 53% | 33% | 2.3 | (0.7, 7.6) | 0.1929 |

Teg-6 = tegaserod 6-mg bid; Plac = placebo; CI = confidence interval; ITT = intent to treat; M + F = males and females

Primary efficacy endpoint: response after 4 weeks of treatment. As a first pass analysis, Fishers Exact tests were performed to evaluate response as a function of genotype. This was done separately for both the 6-mg bid tegaserod and placebo treated groups. Using this criteria alone, significant associations (p<0.05, no multiple test correction) were seen for 4 SNPs in the drug target (HTR4), 1 SNP in the sodium channel SCNN1A, and 2 SNPs in the Na—K-2Cl cotransporter SLC12A2 for the tegaserod treated group (see TABLE 6). An additional SNP in tryptophan 2,3-dioxygenase (TD02) gene (SNP 3746) also demonstrated a significant p-value in the tegaserod group (p<0.01). No statistically significant associations were seen among the placebo treatment group.

TABLE 6
Response as a function of genotype for the 12 SNPs demonstrating differential
response to tegaserod 6-mg bid compared to placebo after 4 weeks of treatment

| Gene | Geno- | Number of Patients | | | | % Responders | | Odds Ratio | | p-value | p-value: R × Geno | |
|---|---|---|---|---|---|---|---|---|---|---|---|---|
| (PG ID) | type | T6 | P | Total (%) | | T6 | P | (95% CI) | | T6 v P | T6 | P |
| All genotypes | | 204 | 204 | 408 | (100%) | 46% | 25% | 2.6 | (1.7, 4.0) | <0.0001 | | |
| AQP3 | CC | 108 | 109 | 217 | (55%) | 43% | 24% | 2.4 | (1.3, 4.2) | 0.0034 | 0.1217 | 0.9627 |
| (1838) | CG | 73 | 76 | 149 | (38%) | 47% | 25% | 2.6 | (1.3, 5.2) | 0.0061 | | |
| | GG | 14 | 11 | 25 | (6%) | 71% | 18% | 11.3 | (1.6, 76.8) | 0.0095 | | |
| HTR3B | AA | 94 | 92 | 186 | (47%) | 46% | 24% | 2.7 | (1.4, 5.0) | 0.0018 | 0.2037 | 0.7217 |
| (3755) | AC | 85 | 83 | 168 | (42%) | 44% | 28% | 2.0 | (1.1, 3.8) | 0.0329 | | |
| | CC | 18 | 25 | 43 | (11%) | 67% | 20% | 8.0 | (2.0, 32.0) | 0.0023 | | |
| HTR4 | CC | 104 | 93 | 197 | (49%) | 42% | 22% | 2.7 | (1.4, 5.0) | 0.0019 | 0.0260 | 0.7182 |
| (1746) | CT | 77 | 91 | 168 | (42%) | 44% | 26% | 2.2 | (1.2, 4.2) | 0.0160 | | |
| | TT | 20 | 19 | 39 | (10%) | 75% | 26% | 8.4 | (2.0, 35.4) | 0.0027 | | |
| HTR4 | AA | 94 | 76 | 170 | (52%) | 43% | 20% | 3.0 | (1.5, 6.0) | 0.0016 | 0.0241 | 0.8938 |
| (3743) | AC | 56 | 64 | 120 | (37%) | 46% | 23% | 2.8 | (1.3, 6.2) | 0.0083 | | |
| | CC | 18 | 20 | 38 | (12%) | 78% | 20% | 14.0 | (2.9, 66.7) | 0.0004 | | |
| HTR4 | AA | 99 | 92 | 191 | (48%) | 43% | 21% | 3.0 | (1.6, 5.6) | 0.0008 | 0.0922 | 0.5387 |
| (3747) | AT | 78 | 85 | 163 | (41%) | 45% | 27% | 2.2 | (1.1, 4.2) | 0.0180 | | |
| | TT | 20 | 21 | 41 | (10%) | 70% | 29% | 5.8 | (1.5, 22.4) | 0.0088 | | |
| HTR4 | CC | 104 | 91 | 195 | (49%) | 43% | 22% | 2.7 | (1.4, 5.1) | 0.0017 | 0.0286 | 0.6431 |
| (3753) | CT | 78 | 89 | 167 | (42%) | 44% | 27% | 2.1 | (1.1, 4.0) | 0.0248 | | |
| | TT | 20 | 20 | 40 | (10%) | 75% | 30% | 7.0 | (1.7, 28.2) | 0.0049 | | |
| HTR4 | AA | 96 | 87 | 183 | (46%) | 46% | 21% | 3.2 | (1.7, 6.3) | 0.0003 | 0.1065 | 0.4487 |
| (3754) | AT | 78 | 89 | 167 | (42%) | 42% | 28% | 1.9 | (1.0, 3.6) | 0.0549 | | |
| | TT | 24 | 24 | 48 | (12%) | 67% | 29% | 4.9 | (1.4, 16.5) | 0.0101 | | |
| HTR4 | AA | 92 | 80 | 172 | (43%) | 39% | 19% | 2.8 | (1.4, 5.6) | 0.0036 | 0.0215 | 0.2558 |
| (3756) | AC | 84 | 94 | 178 | (45%) | 46% | 30% | 2.0 | (1.1, 3.8) | 0.0225 | | |
| | CC | 24 | 26 | 50 | (13%) | 71% | 23% | 8.1 | (2.3, 28.8) | 0.0008 | | |
| MLN | CC | 75 | 58 | 133 | (33%) | 47% | 26% | 2.5 | (1.2, 5.3) | 0.0144 | 0.1131 | 0.8842 |
| (1783) | CT | 97 | 120 | 217 | (55%) | 40% | 25% | 2.0 | (1.1, 3.6) | 0.0170 | | |
| | TT | 27 | 21 | 48 | (12%) | 63% | 19% | 7.2 | (1.9, 27.6) | 0.0026 | | |
| SCNN1A | GG | 34 | 37 | 71 | (18%) | 62% | 24% | 5.0 | (1.8, 14.0) | 0.0015 | 0.0462 | 0.9283 |
| (3806) | GT | 90 | 108 | 198 | (50%) | 47% | 26% | 2.5 | (1.4, 4.5) | 0.0024 | | |
| | TT | 72 | 57 | 129 | (32%) | 36% | 23% | 1.9 | (0.9, 4.2) | 0.1036 | | |
| SLC12A2 | AA | 13 | 19 | 32 | (8%) | 69% | 16% | 12.0 | (2.2, 66.0) | 0.0025 | 0.0030 | 0.6914 |
| (1842) | AG | 51 | 68 | 119 | (29%) | 27% | 25% | 1.1 | (0.5, 2.6) | 0.7640 | | |
| | GG | 139 | 114 | 253 | (63%) | 51% | 26% | 2.9 | (1.7, 5.0) | <0.0001 | | |
| SLC12A2 | CC | 12 | 20 | 32 | (9%) | 75% | 15% | 17.0 | (2.8, 102) | 0.0008 | 0.0017 | 0.3370 |
| (3801) | CT | 45 | 60 | 105 | (28%) | 27% | 30% | 0.8 | (0.4, 2.0) | 0.7096 | | |
| | TT | 129 | 108 | 237 | (63%) | 52% | 22% | 3.8 | (2.1, 6.7) | <0.0001 | | |

Response = mean increase of 1 or more CSBMs/week over baseline; ITT Caucasians with at least 7 days of treatment; PG ID = unique internal SNP identifier; T6 = tegaserod 6-mg bid; P = placebo; CI = confidence interval; % of Pop. = percent of population with that genotype; p-value: R × geno = p-value for test of significant difference between response rates by genotype within each treatment group (note that none of the p-values have been adjusted for multiple testing); genotypes demonstrating the best response are shown in bold.

We also wanted to see if we could identify SNPs that demonstrated both a higher than average response to tegaserod and that maximized the difference in response between tegaserod and placebo treated patients, as determined by odds ratios. Using a cut-off of at least a 60% response rate for tegaserod and an odds ratio of 5 or greater, we identified twelve SNPs of the 55 SNPs that fit these criteria following 4 weeks of treatment. These “high responder” SNPs included six in the drug target HTR4, one in HTR3B, two in the Na—K-2Cl cotransporter (SLC12A2), one in the aquaporin channel AQP3, one in motilin (MLN) and one in the non-voltage gated Na channel SCNN1A (see TABLE 6). It was interesting to note that the “high responder” genotypes for four of the genes (AQP3, HTR3B, MLN, and SLC12A2), in addition to showing higher response rates to tegaserod, also showed a trend towards a lower response rate in placebo treated individuals.

In all cases, it was the minor homozygous genotype that displayed the higher than average response to tegaserod and that for each individual SNP this accounted for only 6-18% of the population. In an effort to increase the number of potentially “high responders”, a further evaluation was performed by grouping individuals into two groups: those that had one or more of these “high responder” SNPs and those that had none. According to this model, approximately half of the patients were labelled as potential “high responders” due to the presence of at least one of the 12 identified SNPs, while the other half had none of the SNPs and thus would be expected to not respond as well. Results of analysis using this 12 SNP model are presented in TABLE 7, along with a breakdown by gender and age.

TABLE 7
Response by genotype after 4 weeks of treatment, comparison of individuals having 1 or more
of the 12 “high responder” SNPs vs. those with none of the SNPs, by age and gender

| Gender | # of the | Number of Patients | | | | % Responders | | Odds Ratio | | p-value | p-value: R × Geno | |
|---|---|---|---|---|---|---|---|---|---|---|---|---|
| (Age) | 12 SNPs | T6 | P | Total (%) | | T6 | P | (95% CI) | | T6 v P | T6 | P |
| M + F | 0-12 | 204 | 204 | 408 | (100%) | 46% | 25% | 2.6 | (1.7, 4.0) | <0.0001 | | |
| (All) | 0 | 105 | 90 | 195 | (48%) | 31% | 27% | 1.3 | (0.7, 2.3) | 0.4672 | <0.0001 | 0.6232 |
| | 1-12 | 99 | 114 | 213 | (52%) | 62% | 23% | 5.4 | (3.0, 9.9) | <0.0001 | | |
| Female | 0-12 | 183 | 181 | 364 | (100%) | 45% | 24% | 2.7 | (1.7, 4.2) | <0.0001 | | |
| (All) | 0 | 95 | 79 | 174 | (48%) | 33% | 24% | 1.5 | (0.8, 3.0) | 0.2143 | 0.0004 | 1.0000 |
| | 1-12 | 88 | 102 | 190 | (52%) | 59% | 24% | 4.7 | (2.5, 8.8) | <0.0001 | | |
| Male | 0-12 | 21 | 23 | 44 | (100%) | 52% | 30% | 2.5 | (0.7, 8.6) | 0.1437 | | |
| (All) | 0 | 10 | 11 | 21 | (48%) | 20% | 45% | 0.3 | (0.0, 2.1) | 0.2278 | 0.0089 | 0.1930 |
| | 1-12 | 11 | 12 | 23 | (52%) | 82% | 17% | 22.5 | (2.6, 195) | 0.0022 | | |
| M + F | 0-12 | 187 | 174 | 361 | (100%) | 46% | 24% | 2.8 | (1.8, 4.3) | <0.0001 | | |
| (<65) | 0 | 98 | 76 | 174 | (48%) | 33% | 25% | 1.5 | (0.7, 2.8) | 0.2727 | 0.0001 | 0.7215 |
| | 1-12 | 89 | 98 | 187 | (52%) | 61% | 22% | 5.3 | (2.8, 10.1) | <0.0001 | | |
| M + F | 0-12 | 17 | 30 | 47 | (100%) | 47% | 30% | 2.1 | (0.6, 7.1) | 0.2473 | | |
| (>=65) | 0 | 7 | 14 | 21 | (45%) | 14% | 36% | 0.3 | (0.0, 3.2) | 0.3173 | 0.0498 | 0.6944 |
| | 1-12 | 10 | 16 | 26 | (55%) | 70% | 25% | 7.0 | (1.2, 40.8) | 0.0267 | | |

Response = mean increase of 1 or more CSBMs/week over baseline; ITT Caucasians with at least 7 days of treatment; PG ID = unique internal SNP identifier; T6 = tegaserod 6-mg bid; P = placebo; CI = confidence interval; % of Pop. = percent of population with that genotype; p-value: R × geno = p-value for test of significant difference between response rates by genotype within each treatment group (note that none of the p-values have been adjusted for multiple testing); genotypes demonstrating the best response are shown in bold. Note that the p-values are likely to biased since they were calculated using the same data used to derive the model.

Using this model, we find that the 52% of the genotyped population with one or more of the 12 SNPs had an overall response rate to tegaserod of 62% compared to 23% for placebo, with an odds ratio of 5.4 (95% CI: 3.0-9.9). This in contrast to the patients without any of the 12 SNPs who showed no significantly different response rates to tegaserod compared to placebo (31% and 27%, respectively), with an odds ratio of 1.3 (95% CI: 0.7-2.3). It was particularly interesting to note that we could also demonstrate a significant response to tegaserod for both the male population as well as for the elderly population for individuals that had at least one of the 12 SNPs, which could not be demonstrated for the study as a whole.

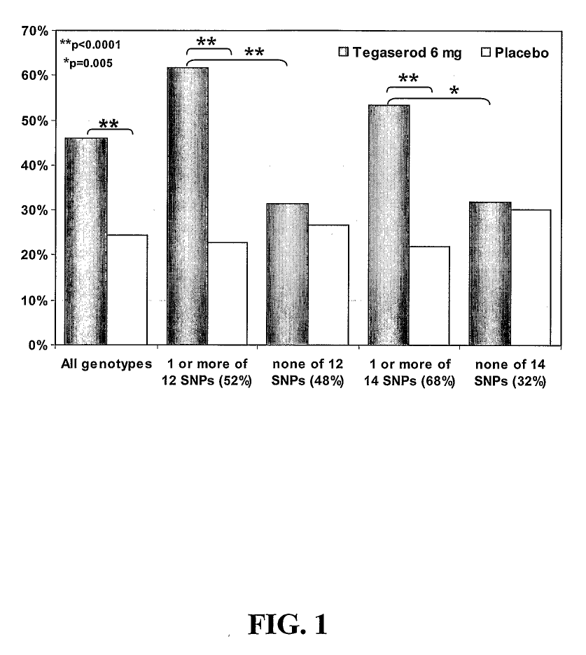

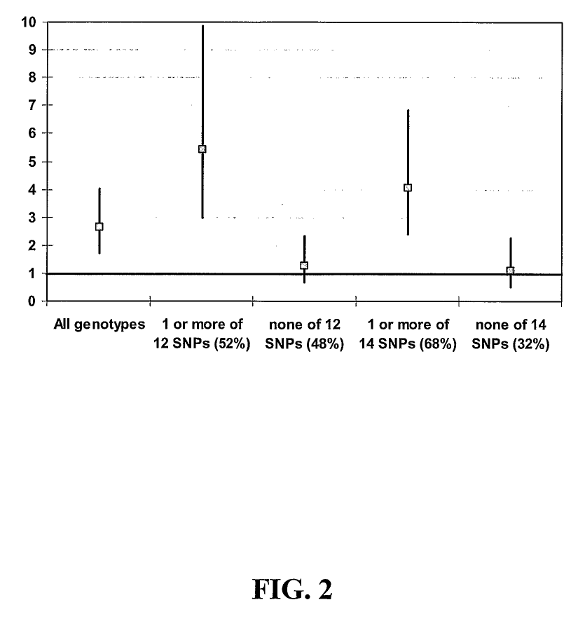

In addition to the 6 genes described above, there was one additional candidate gene (TPH1; tryptophan hydroxylase) that appeared interesting. As shown in TABLE 8, one genotype from each of the two assays for this gene (GG for SNP-1756 and TT for SNP-3784) had an odds ratio of >5. This was primarily due to the low placebo response rate of 15-16% as opposed to a much higher than average response to tegaserod. However, it was also interesting to note that individuals homozygous for the opposite allele had no significant difference in response rates to tegaserod compared to placebo. Also, unlike the 12 “high responder” SNPs described above, for TPH1 it was the major homozygous allele that showed the better overall response, representing 35-36% of the population. Comparisons of response rates and odds ratios categorized by presence or absence of either the original 12 “high responder” SNPs or by the inclusion of the additional 2 TPH1 SNPs are shown in FIG. 1 and FIG. 2, respectively.

TABLE 8
Response by genotype for the TPH1 gene following 4 weeks of treatment

| Gene | Geno- | Number of Patients | | | | % Responders | | Odds Ratio | | p-value | p-value: R × Geno | |
|---|---|---|---|---|---|---|---|---|---|---|---|---|
| (PG ID) | type | T6 | P | Total (%) | | T6 | P | (95% CI) | | T6 v P | T6 | P |
| All genotypes | | 204 | 204 | 408 | (100%) | 46% | 25% | 2.6 | (1.7, 4.0) | <0.0001 | | |
| TPH1 | GG | 76 | 61 | 137 | (35%) | 51% | 16% | 5.4 | (2.4, 12.1) | <0.0001 | 0.3512 | 0.2476 |
| (1756) | GT | 86 | 102 | 188 | (48%) | 48% | 27% | 2.4 | (1.3, 4.4) | 0.0043 | | |
| | TT | 33 | 33 | 66 | (17%) | 36% | 27% | 1.5 | (0.5, 4.3) | 0.4314 | | |
| TPH1 | CC | 34 | 34 | 68 | (17%) | 32% | 24% | 1.6 | (0.5, 4.5) | 0.4209 | 0.1935 | 0.1686 |
| (3784) | CT | 86 | 96 | 182 | (47%) | 49% | 28% | 2.4 | (1.3, 4.5) | 0.0041 | | |
| | TT | 76 | 65 | 141 | (36%) | 50% | 15% | 5.5 | (2.4, 12.4) | <0.0001 | | |

| | | | | | | | | | | | |
|---|---|---|---|---|---|---|---|---|---|---|---|
| 0 of 14 SNPs | 69 | 63 | 132 | (32%) | 32% | 30% | 1.1 | (0.5, 2.3) | 0.8312 | 0.0047 | 0.2214 |
| >=1 of 14 SNPs | 135 | 141 | 276 | (68%) | 53% | 22% | 4.1 | (2.4, 6.8) | <0.0001 | | |

Response = mean increase of 1 or more CSBMs/week over baseline; ITT Caucasians with at least 7 days of treatment; PG ID = unique internal SNP identifier; T6 = tegaserod 6-mg bid; P = placebo; CI = confidence interval; % of Pop. = percent of population with that genotype; p-value: R × geno = p-value for test of significant difference between response rates by genotype within each treatment group (note that none of the p-values have been adjusted for multiple testing); 14 SNPs include the original 12 SNPs plus the 2 TPH1 SNPs; genotypes demonstrating the best response are shown in bold. Note that the p-values are likely to biased since they were calculated using the same data used to derive the model.

While the inclusion of the 2 additional TPH SNPs reduces the overall response to tegaserod for those individuals that do have at least one of the 14 SNPs, compared to the original 12 “high responder” SNPs, it does increase the percentage of patients in that category to 68%. In addition, it further reduces the difference in response rates between treatment and placebo for those 32% of individuals who do not possess any of these SNPs, thus better defining the apparent “non-responder” population.

Secondary efficacy endpoint: response after 12 weeks of treatment. The same comparison as above was performed using the secondary efficacy endpoint of a mean increase of 1 or more CSBM/week over baseline after 12 weeks of treatment. The results for the 12 individual “high responder” SNPs are presented in TABLE 9 and the results using the comparison of individuals with or without at least one of the twelve SNPs is presented in TABLE 10.

TABLE 9
Response as a function of genotype for the 12 SNPs demonstrating differential
response to tegaserod 6-mg bid compared to placebo after 12 weeks of treatment

| Gene | Geno- | Number of Patients | | | | % Responders | | Odds Ratio | | p-value | p-value: R × Geno | |
|---|---|---|---|---|---|---|---|---|---|---|---|---|
| (PG ID) | type | T6 | P | Total (%) | | T6 | P | (95% CI) | | T6 v P | T6 | P |
| All genotypes | | 204 | 204 | 408 | (100%) | 49% | 25% | 2.8 | (1.9, 4.3) | <0.0001 | | |
| AQP3 | CC | 108 | 109 | 217 | (55%) | 47% | 25% | 2.7 | (1.5, 4.8) | 0.0006 | 0.5198 | 0.8270 |
| (1838) | CG | 73 | 76 | 149 | (38%) | 49% | 28% | 2.5 | (1.3, 5.0) | 0.0067 | | |
| | GG | 14 | 11 | 25 | (6%) | 64% | 18% | 8.1 | (1.2, 53.2) | 0.0239 | | |
| HTR3B | AA | 94 | 92 | 186 | (47%) | 49% | 24% | 3.0 | (1.6, 5.7) | 0.0004 | 0.3210 | 0.8318 |
| (3755) | AC | 85 | 83 | 168 | (42%) | 47% | 28% | 2.3 | (1.2, 4.4) | 0.0098 | | |
| | CC | 18 | 25 | 43 | (11%) | 67% | 28% | 5.1 | (1.4, 19.1) | 0.0128 | | |
| HTR4 | CC | 104 | 93 | 197 | (49%) | 44% | 20% | 3.1 | (1.6, 5.8) | 0.0004 | 0.1078 | 0.1237 |
| (1746) | CT | 77 | 91 | 168 | (42%) | 49% | 27% | 2.6 | (1.4, 4.9) | 0.0036 | | |
| | TT | 20 | 19 | 39 | (10%) | 70% | 42% | 3.2 | (0.9, 12.0) | 0.0830 | | |
| HTR4 | AA | 94 | 76 | 170 | (52%) | 48% | 20% | 3.7 | (1.9, 7.5) | 0.0001 | 0.0633 | 0.5601 |
| (3743) | AC | 56 | 64 | 120 | (37%) | 52% | 19% | 4.7 | (2.1, 10.5) | 0.0002 | | |
| | CC | 18 | 20 | 38 | (12%) | 78% | 30% | 8.2 | (1.9, 35.4) | 0.0037 | | |
| HTR4 | AA | 99 | 92 | 191 | (48%) | 41% | 21% | 2.7 | (1.4, 5.2) | 0.0021 | 0.0841 | 0.2391 |
| (3747) | AT | 78 | 85 | 163 | (41%) | 54% | 27% | 3.1 | (1.6, 6.0) | 0.0005 | | |
| | TT | 20 | 21 | 41 | (10%) | 65% | 38% | 3.0 | (0.8, 10.8) | 0.0888 | | |
| HTR4 | CC | 104 | 91 | 195 | (49%) | 45% | 21% | 3.1 | (1.7, 5.9) | 0.0004 | 0.1220 | 0.1772 |
| (3753) | CT | 78 | 89 | 167 | (42%) | 50% | 27% | 2.7 | (1.4, 5.2) | 0.0023 | | |
| | TT | 20 | 20 | 40 | (10%) | 70% | 40% | 3.5 | (0.9, 13.0) | 0.0597 | | |
| HTR4 | AA | 96 | 87 | 183 | (46%) | 50% | 20% | 4.1 | (2.1, 8.0) | <0.0001 | 0.3286 | 0.1340 |
| (3754) | AT | 78 | 89 | 167 | (42%) | 45% | 29% | 2.0 | (1.0, 3.7) | 0.0366 | | |
| | TT | 24 | 24 | 48 | (12%) | 63% | 38% | 2.8 | (0.9, 8.9) | 0.0865 | | |
| HTR4 | AA | 92 | 80 | 172 | (43%) | 39% | 20% | 2.6 | (1.3, 5.1) | 0.0066 | 0.0120 | 0.2648 |
| (3756) | AC | 84 | 94 | 178 | (45%) | 54% | 28% | 3.0 | (1.6, 5.6) | 0.0004 | | |
| | CC | 24 | 26 | 50 | (13%) | 71% | 35% | 4.6 | (1.4, 15.2) | 0.0112 | | |
| MLN | CC | 75 | 58 | 133 | (33%) | 49% | 28% | 2.6 | (1.2, 5.3) | 0.0114 | 0.1253 | 0.4967 |
| (1783) | CT | 97 | 120 | 217 | (55%) | 44% | 27% | 2.2 | (1.2, 3.9) | 0.0066 | | |
| | TT | 27 | 21 | 48 | (12%) | 67% | 14% | 12.0 | (2.8, 51.7) | 0.0003 | | |
| SCNN1A | GG | 34 | 37 | 71 | (18%) | 62% | 22% | 5.9 | (2.1, 16.6) | 0.0006 | 0.2529 | 0.8640 |
| (3806) | GT | 90 | 108 | 198 | (50%) | 49% | 27% | 2.6 | (1.4, 4.7) | 0.0014 | | |
| | TT | 72 | 57 | 129 | (32%) | 44% | 26% | 2.2 | (1.1, 4.7) | 0.0343 | | |
| SLC12A2 | AA | 13 | 19 | 32 | (8%) | 54% | 16% | 6.2 | (1.2, 32.3) | 0.0248 | 0.9165 | 0.5807 |
| (1842) | AG | 51 | 68 | 119 | (29%) | 47% | 25% | 2.7 | (1.2, 5.8) | 0.0126 | | |
| | GG | 139 | 114 | 253 | (63%) | 50% | 28% | 2.5 | (1.5, 4.3) | 0.0005 | | |
| SLC12A2 | CC | 12 | 20 | 32 | (9%) | 58% | 15% | 7.9 | (1.5, 43) | 0.0117 | 0.9079 | 0.5354 |
| (3801) | CT | 45 | 60 | 105 | (28%) | 49% | 28% | 2.4 | (1.1, 5.4) | 0.0318 | | |
| | TT | 129 | 108 | 237 | (63%) | 50% | 26% | 2.9 | (1.7, 5.0) | 0.0001 | | |

Response = mean increase of 1 or more CSBMs/week over baseline; ITT Caucasians with at least 7 days of treatment; PG ID = unique internal SNP identifier; T6 = tegaserod 6-mg bid; P = placebo; CI = confidence interval; % of Pop. = percent of population with that genotype; p-value: R × geno = p-value for test of significant difference between response rates by genotype within each treatment group (note that none of the p-values have been adjusted for multiple testing); genotypes demonstrating the best response are shown in bold.

TABLE 10
Response by genotype after 12 weeks of treatment, comparison of individuals having 1 or more
of the 12 “high responder” SNPs vs. those with none of the SNPs, by age and gender

| Gender | # of the | Number of Patients | | | | % Responders | | Odds Ratio | | p-value | p-value: R × Geno | |
|---|---|---|---|---|---|---|---|---|---|---|---|---|
| (Age) | 12 SNPs | T6 | P | Total (%) | | T6 | P | (95% CI) | | T6 v P | T6 | P |
| M + F | 0-12 | 204 | 204 | 408 | (100%) | 49% | 25% | 2.8 | (1.9, 4.3) | <0.0001 | | |
| (All) | 0 | 105 | 90 | 195 | (48%) | 36% | 30% | 1.3 | (0.7, 2.4) | 0.3619 | 0.0003 | 0.1996 |
| | 1-12 | 99 | 114 | 213 | (52%) | 63% | 22% | 6.0 | (3.3, 10.9) | <0.0001 | | |
| Female | 0-12 | 183 | 181 | 364 | (100%) | 48% | 25% | 2.8 | (1.8, 4.4) | <0.0001 | | |
| (All) | 0 | 95 | 79 | 174 | (48%) | 36% | 28% | 1.4 | (0.8, 2.8) | 0.2656 | 0.0007 | 0.4886 |
| | 1-12 | 88 | 102 | 190 | (52%) | 61% | 23% | 5.5 | (2.9, 10.3) | <0.0001 | | |
| Male | 0-12 | 21 | 23 | 44 | (100%) | 57% | 30% | 3.0 | (0.9, 10.5) | 0.0774 | | |
| (All) | 0 | 10 | 11 | 21 | (48%) | 40% | 45% | 0.8 | (0.1, 4.5) | 0.8055 | 0.1984 | 0.1930 |
| | 1-12 | 11 | 12 | 23 | (52%) | 73% | 17% | 13.3 | (1.8, 100) | 0.0081 | | |
| M + F | 0-12 | 187 | 174 | 361 | (100%) | 49% | 24% | 3.0 | (1.9, 4.7) | <0.0001 | | |
| (<65) | 0 | 98 | 76 | 174 | (48%) | 37% | 26% | 1.6 | (0.8, 3.1) | 0.1457 | 0.0007 | 0.5948 |
| | 1-12 | 89 | 98 | 187 | (52%) | 62% | 22% | 5.6 | (3.0, 10.6) | <0.0001 | | |
| M + F | 0-12 | 17 | 30 | 47 | (100%) | 53% | 33% | 2.3 | (0.7, 7.6) | 0.1929 | | |
| (>=65) | 0 | 7 | 14 | 21 | (45%) | 29% | 50% | 0.4 | (0.1, 2.8) | 0.3613 | 0.1534 | 0.1216 |
| | 1-12 | 10 | 16 | 26 | (55%) | 70% | 19% | 10.1 | (1.6, 64.0) | 0.0104 | | |

Response = mean increase of 1 or more CSBMs/week over baseline; ITT Caucasians with at least 7 days of treatment; PG ID = unique internal SNP identifier; T6 = tegaserod 6-mg bid; P = placebo; CI = confidence interval; % of Pop. = percent of population with that genotype; p-value: R × geno = p-value for test of significant difference between response rates by genotype within each treatment group (note that none of the p-values have been adjusted for multiple testing); genotypes demonstrating the best response are shown in bold. Note that the p-values are likely to biased since they were calculated using the same data used to derive the model.

In general, the findings at 12 weeks were similar to what was seen after 4 weeks of treatment, although the findings were not as statistically significant (for reasons that are not clear). It was interesting to note that while the “high responder” genotypes for the drug target, HTR4, remained higher than average for the tegaserod-treated patients (>60%), this was now also accompanied by a higher than average response rate (30-40%) for the placebo-treated individuals as well. As with the 4-week response, the AQP, MLN, and SLC12A2 “high responder” genotypes continued to demonstrate a lower than average placebo response rate in addition to higher than average response to tegaserod. This lower response to placebo was again seen for the 2 TPH1 SNPs as well, as demonstrated in TABLE 11.

TABLE 11
Response by genotype for the TPH gene following 12 weeks of treatment

| Gene | Geno- | Number of Patients | | | | % Responders | | Odds Ratio | | p-value | p-value: R × Geno | |
|---|---|---|---|---|---|---|---|---|---|---|---|---|
| (PG ID) | type | T6 | P | Total (%) | | T6 | P | (95% CI) | | T6 v P | T6 | P |
| All genotypes | | 204 | 204 | 408 | (100%) | 49% | 25% | 2.8 | (1.9, 4.3) | <0.0001 | | |
| TPH1 | GG | 76 | 61 | 137 | (35%) | 51% | 15% | 6.1 | (2.6, 14.1) | <0.0001 | 0.8439 | 0.0700 |
| (1756) | GT | 86 | 102 | 188 | (48%) | 51% | 30% | 2.4 | (1.3, 4.4) | 0.0039 | | |
| | TT | 33 | 33 | 66 | (17%) | 45% | 27% | 2.2 | (0.8, 6.2) | 0.1276 | | |
| TPH1 | CC | 34 | 34 | 68 | (17%) | 41% | 26% | 1.9 | (0.7, 5.4) | 0.2033 | 0.5798 | 0.0864 |
| (3784) | CT | 86 | 96 | 182 | (47%) | 52% | 30% | 2.5 | (1.4, 4.7) | 0.0025 | | |
| | TT | 76 | 65 | 141 | (36%) | 51% | 15% | 5.8 | (2.6, 13.0) | <0.0001 | | |

| | | | | | | | | | | | |
|---|---|---|---|---|---|---|---|---|---|---|---|
| 0 of 14 SNPs | 69 | 63 | 132 | (32%) | 39% | 35% | 1.2 | (0.6, 2.4) | 0.6184 | 0.0543 | 0.0550 |
| >=1 of 14 SNPs | 135 | 141 | 276 | (68%) | 54% | 21% | 4.4 | (2.6, 7.4) | <0.0001 | | |

Response = mean increase of 1 or more CSBMs/week over baseline; ITT Caucasians with at least 7 days of treatment; PG ID = unique internal SNP identifier; T6 = tegaserod 6-mg bid; P = placebo; CI = confidence interval; % of Pop. = percent of population with that genotype; p-value: R × geno = p-value for test of significant difference between response rates by genotype within each treatment group (note that none of the p-values have been adjusted for multiple testing); 14 SNPs include the original 12 SNPs plus the 2 TPH1 SNPs; genotypes demonstrating the best response are shown in bold. Note that the p-values are likely to biased since they were calculated using the same data used to derive the model.

Analysis by logistic regression. A further analysis was performed using a logistic remodel to take into account gender and age in addition to treatment and “high responder” genotype status. The results of these analyses, using either the 12 SNP or the 14 SNP model are presented in TABLE 12, following 4 or 12 weeks of treatment. While the inclusion of age and gender in each of these models do not appear to have a significant effect on response, the combination of treatment with genotype status does have a highly significant effect on outcome, more so than treatment alone. This effect is slightly more significant using the model containing the original 12 “high responder” SNPs as compared to the model using all 14 SNPs.

TABLE 12
Logistic regression analysis of response using “high responder”
genotype classification, gender, age, and treatment

| Model includes 12 SNPs in 6 genes | | | | Model includes 14 SNPs in 7 genes | | | |
|---|---|---|---|---|---|---|---|
| | | Wald Chi- | | | | Wald Chi- | |
| Effect | DF | Square | Pr > ChiSq | Effect | DF | Square | Pr > ChiSq |

RESP4: increase of 1 or more CSBM/week over baseline after 4 weeks of treatment

| | | | | | | | |
|---|---|---|---|---|---|---|---|
| TRTC | 1 | 3.97 | 0.0463 | TRTC | 1 | 2.62 | 0.1054 |
| AGECAT | 1 | 0.03 | 0.8730 | AGECAT | 1 | 0.12 | 0.7252 |
| SEX1C | 1 | 0.61 | 0.4349 | SEX1C | 1 | 0.53 | 0.4649 |
| HR_12SNPS | 1 | 5.75 | 0.0165 | HR_14SNPs | 1 | 1.08 | 0.2987 |
| TRTC*AGECAT | 1 | 0.38 | 0.5373 | TRTC*AGECAT | 1 | 0.11 | 0.7432 |
| TRTC*SEX1C | 1 | 0.00 | 0.9852 | TRTC*SEX1C | 1 | 0.00 | 0.9674 |
| TRTC*HR_12SNPS | 1 | 10.88 | 0.0010 | TRTC*HR_14SNPs | 1 | 7.81 | 0.0052 |

RESP12: increase of 1 or more CSBM/week over baseline after 12 weeks of treatment

| | | | | | | | |
|---|---|---|---|---|---|---|---|
| TRTC | 1 | 5.65 | 0.0175 | TRTC | 1 | 4.11 | 0.0426 |
| AGECAT | 1 | 0.33 | 0.5663 | AGECAT | 1 | 0.48 | 0.4881 |
| SEX1C | 1 | 0.53 | 0.4677 | SEX1C | 1 | 0.48 | 0.4867 |
| HR_12SNPS | 1 | 2.34 | 0.1260 | HR_14SNPs | 1 | 0.02 | 0.8913 |
| TRTC*AGECAT | 1 | 0.38 | 0.5382 | TRTC*AGECAT | 1 | 0.12 | 0.7310 |
| TRTC*SEX1C | 1 | 0.07 | 0.7969 | TRTC*SEX1C | 1 | 0.04 | 0.8325 |
| TRTC*HR_12SNPS | 1 | 11.79 | 0.0006 | TRTC*HR14_SNPs | 1 | 7.79 | 0.0053 |

TRTC = treatment category (tegaserod 6-mg bid or placebo); SEX1C = gender; AGECAT = age category (< or > = 65), HR_12SNPS = categorization by presence or absence of 1 or more of the 12 “high responder” SNPs; HR_14SNPS = categorization by presence of all 14 SNPs in 7 genes;
*= interaction between 2 parameters; Pr > ChiSq = p-value associated with Chi-square test (significant effect if p < 0.05)

Additional secondary endpoints. In addition to the above assessments, 5 additional secondary efficacy endpoints were evaluated from the weekly diary assessments. These included the weekly changes in satisfaction with bowel habits, bothersome constipation, bothersome abdominal bloating, bothersome abdominal pain, and global constipation relief. TABLE 13 presents the results of analysis comparing the overall change in these parameters at the end of 12 weeks of treatment by treatment (tegaserod 6-mg bid vs. placebo) and whether they had 1 or more of the 12 “high responder” SNPs. An ANCOVA model was applied using the baseline value of these assessments as a quantitative co-variant.

Treatment with tegaserod resulted in better outcome compared to placebo for all patients combined (lower value indicates better response). When evaluated by “high responder” genotype, there was no treatment difference for the individuals with none of the 12 SNPs (“T6 vs. P: 0 SNPs”). However, individuals with 1 or more of the 12 SNPs demonstrated significantly greater responses to tegaserod than placebo for all of the 5 assessments (“T6 vs. P: >=1 SNPs”). Also, within the tegaserod treated population, there were significant differences in outcome between individuals with or without at least 1 of the 12 SNPs for bothersome constipation, global constipation relief, and overall satisfaction with bowel habits. There were no significant differences in the baseline scores between individuals with or without 1 of more of the “high responder” SNPs. Thus these data are consistent with differential response seen for the primary efficacy endpoint based upon number of CSBMs.

TABLE 13
ANCOVA analysis of weekly diary assessments following 12 weeks of treatment; comparison
by treatment and presence of 1 or more of the 12 “high responder” SNPs

| | | | LS Mean Value | | | p-value (ANCOVA) | | | |
|---|---|---|---|---|---|---|---|---|---|
| Efficacy | | | All | 0 of 12 | >=1 of | 0 vs >=1 | T6 vs P: | T6 vs P: | T6 vs P: >=1 |
| Variable | Treatment | N | geno | SNPs | 12 SNPs | SNPs | All | 0 SNPs | SNPs |
| Bothersome | Baseline | 398 | 2.77 | 2.78 | 2.75 | 0.7455 | | | |
| abdominal | 12-wk Placebo | 198 | 2.30 | 2.28 | 2.32 | 0.7722 | | | |
| bloating | 12-wk Teg-6 mg | 200 | 2.09 | 2.18 | 2.00 | 0.1148 | 0.0092 | 0.3769 | 0.0047 |
| Bothersome | Baseline | 398 | 2.34 | 2.34 | 2.34 | 0.9853 | | | |
| abdominal | 12-wk Placebo | 198 | 2.04 | 2.00 | 2.06 | 0.5916 | | | |
| pain | 12-wk Teg-6 mg | 200 | 1.84 | 1.90 | 1.77 | 0.2435 | 0.0132 | 0.3859 | 0.0084 |
| Bothersome | Baseline | 398 | 2.80 | 2.81 | 2.79 | 0.8194 | | | |
| constipation | 12-wk Placebo | 198 | 2.41 | 2.43 | 2.40 | 0.7823 | | | |
| | 12-wk Teg-6 mg | 200 | 2.07 | 2.25 | 1.87 | 0.0006 | <0.0001 | 0.1255 | <0.0001 |
| Global | Baseline | 398 | 2.75 | 2.76 | 2.74 | 0.7709 | | | |
| constipation | 12-wk Placebo | 198 | 2.35 | 2.34 | 2.35 | 0.9002 | | | |
| relief | 12-wk Teg-6 mg | 200 | 2.08 | 2.20 | 1.95 | 0.0139 | 0.0003 | 0.1934 | <0.0001 |
| Satisfaction | Baseline | 398 | 3.09 | 3.11 | 3.06 | 0.6018 | | | |
| with bowel | 12-wk Placebo | 198 | 2.64 | 2.65 | 2.63 | 0.8685 | | | |
| habit | 12-wk Teg-6 mg | 200 | 2.33 | 2.48 | 2.17 | 0.0068 | 0.0003 | 0.1719 | <0.0001 |

LS = least squares mean; N = number of observations; Teg-6 = tegaserod 6-mg bid.

Allele frequencies. TABLE 14 displays the minor allele frequencies for the 12 “high responder” SNPs for the genotyped Caucasian population for comparison with the minor allele frequencies given by ABI for a reference panel of Caucasians. There does not appear to be any large differences in allele frequencies between our chronic constipation population and that of the reference samples.

TABLE 14
Allele frequencies for the 12 ”high responder“ SNPs

| | | Minor Allele Frequency | | |
|---|---|---|---|---|
| PG locus ID | Gene Symbol | Allele | E2302 (Cauc) | ABI ref (Cauc) |
| 1838 | AQP3 | G | 0.25 | 0.28 |
| 3755 | HTR3B | C | 0.32 | 0.26 |
| 1746 | HTR4 | T | 0.30 | 0.32 |
| 3743 | HTR4 | C | 0.30 | 0.30 |
| 3747 | HTR4 | T | 0.31 | 0.30 |
| 3753 | HTR4 | T | 0.31 | 0.33 |
| 3754 | HTR4 | T | 0.33 | 0.36 |
| 3756 | HTR4 | C | 0.35 | 0.31 |
| 1783 | MLN | T | 0.39 | N/A |
| 3806 | SCNN1A | G | 0.43 | 0.48 |
| 1842 | SLC12A2 | A | 0.22 | 0.22 |
| 3801 | SLC12A2 | C | 0.23 | 0.22 |

ABI ref = minor allele frequencies from Applied Biosystems Inc. reference samples; Cauc = Caucasian; E2302 = current genotyping study (CHTF919E2302); N/A = not available for Caucasian population

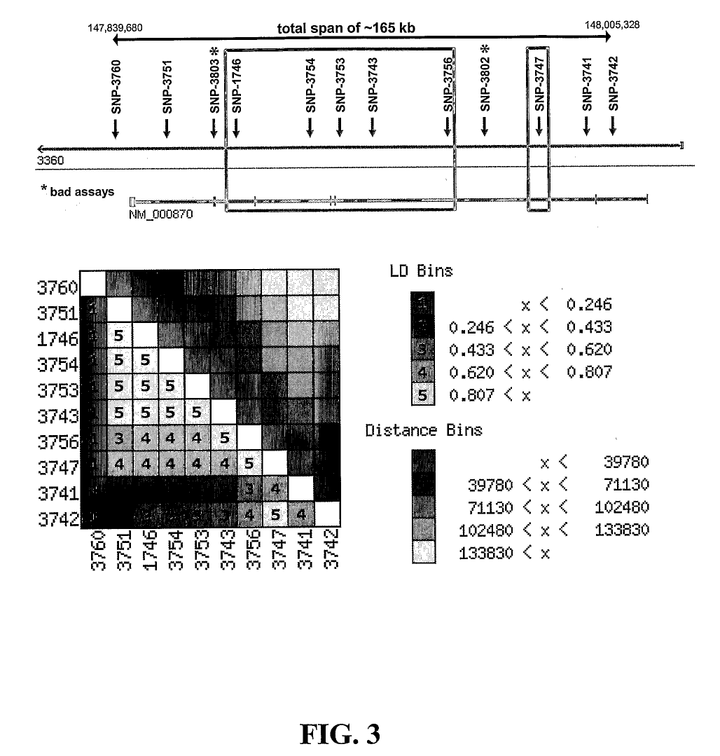

Evaluation of linkage disequilibrium. Of the 12 “high responder” SNPs, 6 of these were located within the drug target HTR4. All of these SNPs were located in intronic regions, spanning nearly 100 kb of genomic DNA (see FIG. 3) Further analysis demonstrated that these 6 SNPs were all in very close linkage disequilibrium, with D′ values of >0.8 for 4 of the SNPs (1746, 3754, 3753 and 3743) and D′>0.6 for SNPs 3756 and 3747 (see FIG. 3). The 2 “high responder” SNPs for the Na—K-2Cl cotransporter SLC12A2 were also in very close LD to each other, as were the 2 TPH1 SNPs.

Details on genes associated with differential response to tegaserod compared to placebo. The initial SNP “high responder” genotype model was based upon 12 SNPs that were present in a total of 6 different genes: 6 in HTR4, 2 in SLC12A2, and 1 each in HTR3B, MLN, AQP3 and SCNNLA. The second 14 SNP model included an additional 2 SNPs in the TPH1 gene. Further details on each of these genes along with their possible role in constipation or response to tegaserod are discussed below.

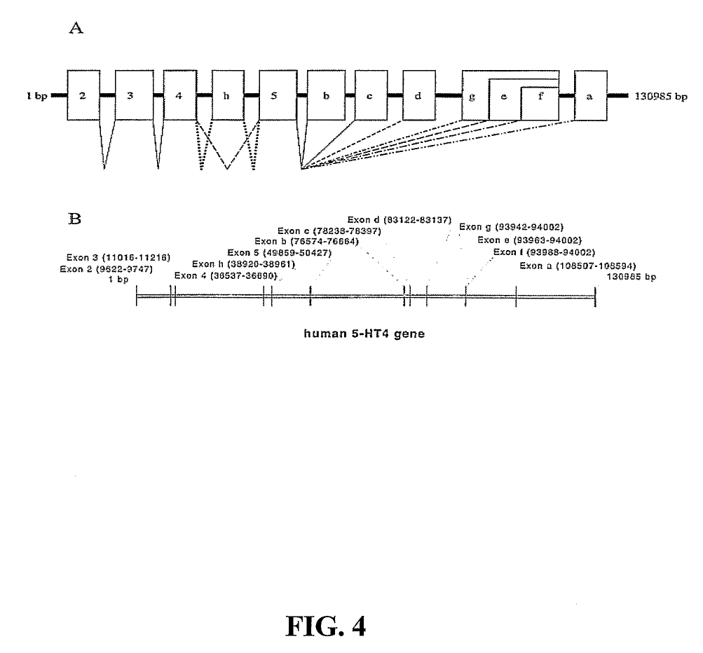

HTR4: 5-HT₄ receptor (drug target). HTR4 is a 7-transmembrane domain G-protein coupled receptor coupled to adenylate cyclase that is prevalent throughout the GI tract as well as a number of other tissues such as the brain. Several variants of the expressed 5-HT₄ receptor have been identified due to alternative splicing of a number of different exons located primarily at the 5′ end of the gene (see FIG. 4). With the exception of exon h, which encodes for a 14 amino acid insertion between transmembrane domains 4 and 5, the alternative spliced exons a-g result in proteins that differ only at the C-terminal domain. Bender E et al., Neurochem. 74(2): 478-89 (2000). The different HTR4 splice variants demonstrate different tissue specificity as well as differences in physiological activity. Bender E et al., Neurochem. 74(2): 478-89 (2000); Blondel O et al., J. Neurochem. 70(6):2252-61 (1998); Claeysen S et al., Mol Pharmacol; 55: 910-920 (1999).

Using FIG. 4 as a reference, the 6 “high responder” HTR4SNPs are located in the following locations: SNPs 3753, 3743, 3756, and 3747 are located downstream of exon 2 in intron 1; SNP 3754 is located between exons 3 and 4; and SNP 1746 is located between exons 4 and 5, upstream of exon h. While none of these SNPs are located in the coding region or directly in the splice sites, it is likely that they are in close LD with another as yet unidentified SNP that does have a functional significance. Due to the large region of linkage disequilibrium between these SNPs (see FIG. 3), it is not possible to predict where this proposed functional SNP would be located. Depending on the location, a number of functional consequences are possible, such as: alterations in the promoter region of the gene which could affect overall expression; alterations in splice sites which could affect which splice variant is expressed; or possibly a change in the amino acid sequence of the protein which could affect the activity of the expressed protein. Additional research would be necessary to determine which, if any, of these scenarios is true.

HTR3B: 5-HT₃ receptor B. HTR3B encodes the B subunit of the 5-HT₃ receptor, a member of the Cys-loop family of ligand-gated ion channels which includes the nicotinic acetylcholine (nAch), GABA_{A} and glycine receptors. Reeves D C & Lummis S C, Mol. Membr. Biol. 19: 11-26 (2002). At least three distinct 5-HT3 receptor subunits are known (A, B, and C), and the functional receptor is an oligomer containing five of these subunits put together, usually a combination of A and B. However, a functional homopentameric receptor composed of only 5-HT_{3A} subunits has also been described. Each subunit consists of four transmembrane domains with evidence the pore of channel is formed by the M2 domain. This receptor acts as a relatively non-selective cation channel and causes fast, depolarizing responses in neurons after activation.

One SNP in HTR3B was genotyped (SNP 3755) and was associated with differential response to tegaserod. The SNP encodes a missense mutation in exon 5 which results in an amino acid change from tyrosine to a serine at amino acid position 129, which is located in the extracellular N-terminal domain. A study has demonstrated that several tyrosine residues in the extracellular domain of the 5-HT_{3A} receptor subunit play important roles in the ligand binding or gating of the channel, as well as in the receptor assembly and structure. Price K L & Lummis S C, J Biol Chem. 279(22):23294-301 (2004). This SNP is also displays high linkage disequilibrium with the 3′ region of the gene that encodes for the transmembrane domains (SNP Browser Version 1.0, Applied Biosystems Inc.).

MLN: motilin. Motilin is a peptide hormone which is mainly secreted by the endocrine cells of the small intestine to act as a regulator of gastrointestinal contraction. The human motilin gene (MLN) consists of five exons and encodes a 115-amino acid preprohormone that includes the 22-amino acid motilin hormone peptide, a 25-amino acid signal peptide, and C-terminal motilin-associated peptide (MAP). Exons 2 and 3 encode the signal peptide and the 22-amino acid motilin peptide, while the C-terminal MAP is largely encoded by Exons 3 and 4. Daikh D I et al., DNA. 8(8):615-21 (1989). The motilin SNP that was associated with higher response in the present analysis (SNP 1783) is a missense mutation that replaces a valine with an alanine at amino acid position 15 of the preprohormone. This places the mutation in the signal peptide region of the gene, position −11 relative to the active motilin peptide. Interestingly, this polymorphism is conserved across species. Depoortere I et al., Peptides. 18(10):1497-503 (1997). Alterations in signal peptide sequences could affect the subcellular localization of the protein, which could affect the secretion of the active motilin peptide, or perhaps prevent the proper separation of the active peptide from the precursor altogether. Altered levels of secreted motilin in plasma have also been observed in patients with idiopathic constipation [Sjolund K et al., Scand J. Gastroenterol. 21(8):914-8 (1986)] and in slow-transit constipation [Peracchi M et al., Scand J. Gastroenterol. 34(1):25-8 (1999)]. An association between the motilin signal peptide polymorphism has been reported in patients with inflammatory bowel disease. Annese V et al., Dig. Dis. Sci. 43(4): 715-9 (1998). However, associated levels plasma motilin were not reported.

AQP3: aquaporin 3. Aquaporin 3 (AQP3) is a member of the aquaporin family of water channels. Fluid secretion and absorption are primary functions of the gastrointestinal tract and aquaporins, with their ability to transport water across epithelia, play a key role in this process. AQP3 is expressed in a number of tissues including the gastrointestinal tract, kidney, liver, pancreas, lung, peripheral leukocytes, spleen, and prostate. Ishibashi K et al., Genomics 27:352-354 (1995). In addition to its water channel function, aquaporin 3 has been found to facilitate the transport of nonionic small solutes such as urea and glycerol, but to a smaller degree. In the rectum, AQP3 may play a key role in regulating the amount of fecal dehydration, thus affecting the hardness or softness of the stool, one of the symptoms associated with chronic constipation. Kierbel A et al., Pflugers Arch. 440(4):609-18 (2000).

The AQP3 gene consists of six exons spanning approximately 6 kb, with the coding region distributed among each of the six exons. Inase N et al., J. Biol. Chem. 270(30): 17913-6 (1995). The AQP3 SNP associated with higher tegaserod response (SNP 1838) is located in intron 1. No functional significance has been associated with this SNP, although it could be in linkage with an as yet undefined functional SNP near the region of exon 1. There is little linkage disequilibrium displayed along the length of the gene, so it is unlikely to be associated with a known functional mutation located at the splice junction of exon 5 and intron 5. Roudier N et al., J Biol Chem. 277(48):45854-9 (2002). This mutation results in a premature stop codon in the translated gene which then codes for a non-functional protein. Interestingly, while individuals that were homozygous for this allele (AQP3ₙᵤₗₗ) did demonstrate reduced glycerol transport in their red blood cells, there were no other reported clinical syndromes related to this AQP3 deficiency, at least under normal conditions.

SLC12A2: Na—K-2Cl cotransporter 1 (NKCC1). SLC12A2 is a member of the solute carrier family that functions to transport sodium, potassium and chloride together across the cellular membrane in an electroneutral fashion. This Na—K-2Cl co-transporter, also known as NKCC1, is expressed in a variety of cell types, both epithelial and non-epithelial. Russell J M, Physiol Rev 80:211-276 (2000). In epithelial cells, such as in the large intestine, NKCC1 is located on the basolateral membrane and serves primarily to provide the cell with the chloride that will be secreted from the apical side. The epithelial Cl secretion accounts for mucosal surface hydration, which has a large impact on the fluid composition of intestinal tract. In non-epithelial cells, the NKCC1 protein acts as key player in regulating cell volume. Lytle C, J. Biol. Chem 272:15069-15077 (1997).

NKCC1 was originally cloned from the shark rectal gland [Xu J-C et al., Proc. Natl. Acad. Sci. USA 91:2201-2205 (1994)] and then shortly thereafter the human gene was cloned from a colon-derived cell line. Payne J A et al., J Biol Chem. 270(30):17977-85 (1995). The expressed protein consists of 1212-amino acids with 12 transmembrane domains that are flanked by intracellular N- and C-terminal domains. Hebert S C et al., Pflugers Arch. 447(5):580-93 (2004). While no human disease has been directly linked to defects in the NKCC1 gene, mutations in this gene have been associated with deafness [Dixon M J et al., Hum. Mol. Genet. 8(8):1579-84 (1999)] and failure of spermatogenesis [Pace A J et al., J. Clin. Invest. 105(4):441-50 (2000)] in mice. Here, we identified two SNPs in this gene that were associated with higher response to tegaserod: SNP 3801, located in intron 2, and SNP 1842, located at the opposite end of the gene in the 3′ UTR.

SCNN1A: non-voltage gated sodium channel, alpha. The SCNN1A gene encodes the alpha subunit a non-voltage gated, amiloride-sensitive sodium channel, commonly known as the epithelial sodium channel or ENaC. This channel is one of the primary routes for sodium absorption in distal colon, and thus plays a key role in maintaining the fluid composition of bowel. While the alpha subunit alone is able to form a functional channel that conducts sodium, in vivo a functional channel is composed of combination of alpha, beta, and gamma subunits.

The SCNN1A gene consists of thirteen exons spanning 17 kb on chromosome 12p13, with the coding region starting in exon 2 and ending in exon 13. Ludwig M et al., Hum Genet. 102(5):576-81 (1998). The “high responder” SNP 3806 is located near the middle of the SCNN1A gene in intron 5. Again, there is a high degree of linkage disequilibrium across the entire gene, so the causative SNP could be anywhere. Interestingly, the two other SCNN1A SNPs which were also genotyped, one near the 5′ end and one near the 3′ end, also showed a somewhat higher response to tegaserod (odds ratio >3.5) and were in LD with SNP 3806. A number of mutations in the SCNN1A gene have been associated with human disease, mainly due to effects in the kidney. Several allelic variants have been associated with type 1 autosomal recessive pseudohypoaldosteronism in several different families (see entry in OMIM #600228: sodium channel, nonvoltage-gated 1, alpha subunit; SCNN1A http://www.ncbi.nlm.nih.gov/entrez/dispomim.cgi?id=600228). An association has also been made between hypertension and a SNP in SCNN1A, presumably due to a lower level of sodium reabsorption in the kidney. Iwai N et al., J. Am. Soc. Nephrol. 13:80-85 (2002). The effects of mutations on this sodium channel in the distal colon, which is also under aldosterone control, could certainly lead to deleterious effects on fluid and electrolyte balance in the bowel.

TPH1: tryptophan hydroxylase 1. Tryptophan hydroxylase is the rate-limiting enzyme in the biosynthesis of serotonin, catalyzing the monooxygenation of tryptophan to 5-hydroxytryptophan (5-HT), which is subsequently decarboxylated to form serotonin. TPH1 is mainly expressed in the periphery, including the intestinal enterochromaffin cells, while the recently identified TPH2 gene is preferentially expressed in the brain. Walther D J et al., Science 299(5603):76 (2003). Serotonin is causally involved in multiple central nervous facets of mood control and in regulating sleep, anxiety, alcoholism, drug abuse, food intake, and sexual behaviour, while in the peripheral tissues, serotonin is involved in regulating vascular tone, gut motility, primary haemostasis, and cell-mediated immune responses. Veenstra-VanderWeele J, Anderson G M & Cook E H Jr., Eur J. Pharmacol. 410(2-3):165-181 (2000).

Two TPH1 SNPs were associated with an odds ratio of >5.0, which was primarily due to a lower response in the placebo group as opposed to a much higher response rate in the tegaserod group. These two SNPs were located in intron 1 (SNP 1756) and in intron 5 (SNP 3784), and are not believed to cause functional mutations themselves. This entire gene is in strong linkage disequilibrium, so again it is likely that the identified SNPs are in linkage with a functional SNP, which could be located anywhere within the gene. A number of studies have evaluated TPH1 polymorphisms and the risk of suicide or other mood disorders with mixed results. SeeArango V et al., J. Psychiatr. Res. 37(5): 375-86 (2003) for a review. The SNPs evaluated in these studies were not located in the coding region. Arango et al. report that no functional SNP has yet been identified. While less has been reported regarding the association of TPH1 genotypes and bowel disorders, a recent report by Coates and colleagues noted that there was a significant reduction in TPH1 messenger RNA in patients with ulcerative colitis, irritable bowel syndrome-constipation predominant (IBS-C) and irritable bowel syndrome-diarrhoea predominant (IBS-D). Coates M D et al., Gastroenterology. 126(7):1657-64 (2004).

Conclusions. The purpose of this pharmacogenetics analysis was to evaluate whether polymorphisms in the candidate genes relating to 5-HT4 and 5-HT3 receptors, serotonin, intestinal secretion or motility were associated with a differential response to tegaserod treatment in patients with chronic constipation. A comparison of response by genotype identified twelve SNPs in six genes that associated with differential response to treatment, with response rates to tegaserod 6-mg bid of >60% and odds ratios of 5 or greater (compared to placebo) after four weeks of treatment (see TABLE 6). This was in comparison to the average overall response rates of 46% and 25% for tegaserod and placebo, respectively, with an odds ratio of 2.6. Using these twelve “high responder” SNPs as one embodiment, we found that individuals that at least one of the twelve SNPs (approximately 50% of the population) had a significantly higher response to tegaserod compared to placebo (62% vs. 23%, odds ratio=5.4). This is in contrast to the remaining approximately 50% of patients without any of the twelve SNPs who showed no significant difference in response to tegaserod compared to placebo (31% vs. 27%, odds ratio=1.3). Results using the twelve SNP model were similar after twelve weeks of treatment, although there were some differences seen at the individual genotype level.

A second embodiment using genotypes from fourteen SNPs to classify patients was also found to be useful to further define the non-responder population. In this embodiment, which included two SNPs from the TPH1 gene in addition to the original twelve “high responder” SNPs, we find that the 32% of patients that had none of the fourteen SNPs had response rates of 32% and 30% to tegaserod and placebo, respectively, with an odds ratio of 1.1. However, using this model decreases the overall response rates to tegaserod for the 68% of the patients that do have at least one of the fourteen 14 SNPs to only 53%, which is lower than the 62% response rate seen using the twelve 12 SNP model.

The identified genes display a wide range of different functions, though all are important in maintaining the normal function of the gastrointestinal tract. The serotonin receptors HTR4 and HTR3B are primary targets of serotonin and thus mediate the downstream effects of this important neurotransmitter, including the regulation of motility, intestinal secretion, as well as visceral sensitivity. TPH1, being the rate-limiting enzyme in the synthesis of serotonin, has an obvious key role in these serotonin-mediated regulatory functions as well. Solute transport is necessary for maintaining the balance of water and electrolytes in the GI tract and thus can have a significant effect on intestinal secretion and absorption. Three of the genes identified play important roles in solute transport: SLC12A2 (NKCC1) is one of the main transport pathways for the secretion of chloride; SCNN1A (ENaC alpha) is important in the absorption of sodium; and AQP3 plays an important role in the transport of water and glycerol. Finally, motilin (MLN) is one of the key gut hormones found to regulate motility of the GI tract.

Based on these findings, chronic constipation may result from a variety of pathophysiological mechanisms related to variants in the above identified genes, all of which respond well to treatment with tegaserod. Patients without these variants do not respond to treatment significantly more than they do to placebo, which could indicated that their chronic constipation is not due to pathophysiological mechanisms but rather to environmental or possibly psychological factors. Interestingly, patients with these variants are also less likely to respond to placebo, again implying that these variants are associated with a true pathophysiology.

All references cited herein are incorporated herein by reference in their entirety and for all purposes to the same extent as if each individual publication, patent, or patent application was specifically and individually indicated to be incorporated by reference in its entirety for all purposes.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled.

SEQUENCES

| SEQ. | | | |
|---|---|---|---|
| ID | PG | | |
| NO. | ID | GENE | Sequence surrounding [SNP] |
| 1 | 1746 | HTR4 | TCTAGATAAGGCGGGAACTGTCATAAGCATGACT |
| | | | ATCAAAGCTAAATAAAACATACTGAATTAAATAC |
| | | | AAAATATGAAGATTAAGTAAAATATTCTTTGAAA |
| | | | GATTGGAAAGTAACCAAGACAAATAAGACTTAGT |
| | | | AGCTACACTCTCAAAAAGAAAAAGAAGACCTCTA |
| | | | CTATTTC [C,T] |
| | | | TTTGGGGCCTTTACTGAGTCATATAAGGTCTGTG |
| | | | TAAAAGTAATTGTGGTTTTTGCCATT |
| 2 | 3743 | HTR4 | AATCTGCTGTCAATACCCTAGGAGAGGGCTTCCT |
| | | | TTCATCTTCTCCTAGACTATCACAACCACTTTTA |
| | | | TCCCACTAACTACTTTGTCTTTGTCTTCAAATTC |
| | | | TTAGTATATGAGACAGCTTAAATTTAACATGTTC |
| | | | AAAATTGAACTCCTGATTTCCCTAACCTCAAACC |
| | | | TGCTTCCCATATGAATACATAGCAAATCTATTTC |
| | | | TCCAGTTGCTCAGGTTAAAAATTTGGAAGCCACT |
| | | | TTTACCAGGTCTCTACCTCTTCTACCTCCAGTAC |
| | | | ATCAGCAAATCCTGATAGCTCTAACTTC [A,C] |
| | | | TAACCTTTCCTGATCCCAACCCTTTACCAGCCAT |
| | | | TCAATGTCATACCATTCTAGTTCCTACTACATCA |
| | | | TGCTTCACTTGGTGACTGCAAAGTCCTCTCAACT |
| | | | GGCATCCCTGCTTCCATCATTTCTCACATACAAT |
| | | | CCACTCTTCACCCACTAGACAGAATGAGCTTTGA |
| | | | AAAATATTGAGATTGGAACATATCACTTCCATAC |
| | | | TCACTGCCCTTAAGATGGCTTGTCATCTCACTAG |
| | | | GAGTAAACCCAGAACCCTCACCATGACCTATGTA |
| | | | AATTTACAAAGCTGGCCACTGGCTGCTT |
| 3 | 3747 | HTR4 | GGGTTACAGGTTGGCAGCAGCTGTGTTCCTGATC |
| | | | TCTTTGTGGGATCTGATAATAACTCTCTCCCCTC |
| | | | GGCCCTTCAGGCACAAGGATGATAACAGCTCACT |
| | | | GCTGTGCTAAGCTCCAAGGTATTCCACCATCCTT |
| | | | TGTTGATTTTAACCTTGCACACACCTTTAACTCA |
| | | | TTTGTGCCTGCTTTCCCTTTCCTATGGGACCTCC |
| | | | TATCAGGCTGCTCAACCTATCACTGTTCCTTCAT |
| | | | GTCAACCACAAATTTGGTTAGCCGTTAAAGTCTT |
| | | | CATTCAAGAAATGGATGTGAATATTTAA [A,T] |
| | | | GTTCAAAGCTAAGGGCCACTGTCAATTTAATCCA |
| | | | AATTGCCTGTTATTATGACCTAGGCTTTGGTGAC |
| | | | CACAGAAGTATACTGAGGATTGTGACACATACAG |
| | | | CCGATTCCAATCGCTTTCATTTTGGGTTGATTTA |
| | | | GTAGGGTATATCTGGAATATATTCTGTGAAGGTT |
| | | | ATATTTTAGTGGAGATGATCTTGATTCTCTAGTA |
| | | | GGATTGAAAAGAAAGTGAATGAAGAAAGGAAAGA |
| | | | AGAAAGTAAGAAAAGAATTGAGGGAGGAAAGAAG |
| | | | GAAGAGAGAGAAAAAAGAAAAAGGACCT |
| 4 | 3753 | HTR4 | CCTGTGCCCCGACAGGCCATGGGTTAGACAAGTT |
| | | | TGTCCTAGACCCAAACCCCACATTTGAATATGGG |
| | | | AAAACCAGGGCCCAGAAAAGAAGAGCTGACTTGT |
| | | | CAGAGCGTGCTAGTGGCTGGTCTGGGAGAGGCTG |
| | | | CTTGTCCTGTGAGCCCACCACTGAGAACACACAG |
| | | | TTCCAAAAATCAAGAATTCTCTGTCTTTCCTGAG |
| | | | CTCTTTTGACTGAAAATTAAGGTGCATGCCCTCT |
| | | | CCAGATGGAAGCAGCCTGCAAAGTCATGGCTTTG |
| | | | GCCTTGTCCAGAACTTCTGTTAAGCTGG [C,T] |
| | | | ACAGAAGACAAGGGGCCAAAGGGAATGAGGAACC |
| | | | AGTCTGGCAGCAGTTGGAGAATGAGAAAGTCCCT |
| | | | CCATTCAGCTGAGAAGTATTTTTATAGGCAGATT |
| | | | GATAGATTGTTTATGACTGAAGTTCAGATGAAAA |
| | | | GAAATTCCAGATATGCTTATTTAAATCACCCTTC |
| | | | TACACTTAAGTTCTAACTATCAACAGTACAAATA |
| | | | TCAGATAGGGAAGATTTAATTCAATGAGGAAAAC |
| | | | TGTACACTTAGAAAAAAGACCTTAATAGGCTGAT |
| | | | GTGGCCACCAAAAATTCATTTGATCTTA |
| 5 | 3754 | HTR4 | AAATATTATGTACAAAGCCTGAACCTGCCCTAAA |
| | | | AGAGTATGTGAGAAGGAGAAACTTTTAATATACT |
| | | | TTCCTTGTGAAATGCATTTCAATCCTATTGGGAA |
| | | | AACAAATGTAATTGTGTGACACAGTTCACAATGA |
| | | | CAGACACTTTTCATTTATACATTACAAAATCGAA |
| | | | TTATACTCCAGGGAGATCATTAAGAAAATGCAGA |
| | | | TACATTTATCTCCAGCACAAGATTAAACATGCAA |
| | | | CCTTGAAGTAAGAATTGTATGTTTTACAAGTAAA |
| | | | CCTGTTTAAAAGAGGATCGGAGACTAAG [A,T] |
| | | | AACTCTTGGAGAATTTTGCCGATAAAACACATCA |
| | | | AATTTTTGAGCTGTGCAGAGGAAGGGTGACAATT |
| | | | CAGCTGGGTGTATCCAAAAAAGAATCTTGCAGTA |
| | | | CTATAGAGAATACTATCTTCAAGGAAGTGAAATT |
| | | | GACTTTTTTTTTAAAAAGGGAATTACATTTAATG |
| | | | CTAAGTAAAGGACAAAGTACAGCATGTTCAGAGA |
| | | | AAGGGGATAAAAATGGTTAAAAGTCTGAAAAGTA |
| | | | TGGCTTATGAGGCCAGCAGAAGGATCTAGAAATA |
| | | | TTTGGCCTAATATAGGGAAGACTTCGGG |
| 6 | 3756 | HTR4 | TGTGTCTGTATCTTTTCTGTCACTTTATATAACT |
| | | | AATTACTGTTTACTCTTTCCAAGCAGGGTGTGTG |
| | | | GGGATCCAAACTGTGGGAGAGAATGGTGTTACTT |
| | | | TGGGGAAGCCTTTGCAGAAACAAGACAGTCTGGA |
| | | | AGAAAGGGAAACTCCAGCTGCAGAATTTTCTTCT |
| | | | CTTTTCTTGCTGTGGCCCCATCCTCCCACCTGGC |
| | | | TCTAGGGGAAAGTCCTATTTAAAAGTGAGAGTTA |
| | | | GTAACTTAGCATCTTTCAGCAGATATCACAGTTG |
| | | | CCTCTGGCACAGCCTCTTGCACCCTCGT [A,C] |
| | | | CTTCCTCTCCTGGCTGTGCCTGCCTCAGCATGGG |
| | | | GATAACTGACTGCCCTCAGTTTTCCTCACCCTGC |
| | | | TACTAGATTTGTAGCTGGATTTGGGGACCTCTGA |
| | | | AGGAATTTTAACCACATAGGGAAGCGGTATGATC |
| | | | AAGTGGAAGAAATGAAAACACCAAGGCTCGTAAG |
| | | | ATTGAGATCTAATCCTGCTCTGCCACCAACCAGC |
| | | | TGAGTCTCCCCTGCTGAACCATCAGTCAGCTCTG |
| | | | TGGTTGCAAGACCGATAGACTTTTCTCAGCACTG |
| | | | GTCTCATTTAATCTCTGCAACACTTGCA |
| 7 | 3755 | HTR3B | AGAAGTGCCATGTGTTGACATTATTTAAGATACA |
| | | | GATCTGGTTACTAATGTTGAGGTTACTCATTATG |
| | | | TCTGTGTTGTTATAGCAGCAAATGAGTTAACTTA |
| | | | CGAGGCTGTCACTGAAAAGCTCATCTTTGCCAGG |
| | | | GTGAATCATCTCATGGAAAATGCGATTCTGTTTT |
| | | | GCAGGGCTAGGCTGGTCCTGGACCTCATGGTCAC |
| | | | TACCATCTCCTAATCAGCCTATGTTTTGAAATGA |
| | | | CCAACATCCTCTCTGTGACAACAAGTTCTCTTGT |
| | | | GTTTCATATAGTGTGGACATTGAAAGAT [A,C] |
| | | | CCCTGACCTTCCCTATGTTTATGTGAACTCATCT |
| | | | GGGACCATTGAGAACTATAAGCCCATCCAGGTGG |
| | | | TCTCTGCGTGCAGTTTAGAGACATATGCTTTTCC |
| | | | ATTTGATGTCCAGAATTGCAGCCTGACCTTCAAG |
| | | | AGCATTCTGCATACAGGTAAACCATGAGAGATAC |
| | | | CCATTAATGCTAGGTTGGTGCACATAGGTCAAAT |
| | | | GATATTATACTATCCTTCAGGTCTATTTTATTCT |
| | | | TGCAGATAATTGGCTATTTAAAAATTGGAATCTC |
| | | | TTCTTGCGGTTTTTTGGCTCCTGCTGTA |
| 8 | 1783 | MLN | CATTGTCCAGCTCCAAGATGGTATCCCGTAAGGC |
| | | | TGTGGCTGCTCTGCTGGTGGTGCATG [C,T] |
| | | | AGCTGCCATGCTGGCCTCCCAGACGGAAGCCTTC |
| | | | GTCCCCATCTTCACCTATGGCGAACT |
| 9 | 1838 | AQP3 | TGTCCCGCTGCGGGGAGATGCTCCACATCCGCTA |
| | | | CCGGCTGCTCCGACAGGCGCTGGCCGAGTGCCTG |
| | | | GGGACCCTCATCCTCGTGGTGAGTGGAGGGAGCC |
| | | | GGGGAAGCCCTTCTCTCTCCAGCCCTTGCACTCC |
| | | | CCAAACTCTCACTTCCCCGAAGGGGCTGTGTTTT |
| | | | CCAAGGTAGCCTGGACCCACCTCCCCAGCT |
| | | | [C,G] |
| | | | TGACCCCCACGCTTAACCGCGGAGGATCAAGCTG |
| | | | ACTTCCAAAGTCCTCTTCCCCACGGTTCTAACCC |
| | | | CCTCTCTGACAGCTCCGACTCTTGCCAGAATGAC |
| | | | AGCTGTTACTCCCCAGTGATAGTGCCGATCGTTT |
| | | | ACCCTCCCACAAGTGACTCAGCCAACAGGCTGGG |
| | | | GGCAGCGGTCACGCTTGCAGTCTGGGACAG |
| 10 | 1842 | SLC12A2 | TGCTATTCAAGTAGCAAAGGAAAACTACTCTCAC |
| | | | AAACTTCAGTTCAACA [A,G] |
| | | | AGAAGAATCACCATTAAGATTGAGATATGGAATT |
| | | | GACTAAAACCGAAGTC |
| 11 | 3801 | SLC12A2 | GTCAAGGGTGGAATTGCCGAGGAATGTTAACTTA |
| | | | ATCTCTCAAAAGTTTGTAGCGGGTTTGGCTAGTT |
| | | | ACGTGATACCGGAGGGCTGCCTCTAACAACCTTC |
| | | | CCCATCCAGTTAGGTATCTCGTGTGCACTTTCTT |
| | | | TCCCACCCACGCTCAACAGTCACCA [C,T] |
| | | | CCCTCTGAATGACAGTAGTGTTTGTGGGCCCTTT |
| | | | AGAGGAGAATGTGCAGTGAGGATCTCTCGAGAGA |
| | | | GGTTGGAGAGCACCTACCATCTGTGTCGTTTTGA |
| | | | AAGATTGTGTTGTGTGGCTTATGTACGCCTATTA |
| | | | GGGATCGCAAGAGTGGGAATGTTGCTGTTTGAAG |
| | | | GAAAACCTTAACAGGGTGTGTTTATGGTCTATGA |
| | | | GTTATTAAAGCTCAATTCTTGCAATCTTGAATAT |
| | | | ACAAAGGATCATAGAGATCTTGTCTTGGGACACG |
| | | | CTGTTCCTAATAATGGCATAACTCTTTTTTTTTG |
| | | | CGATGAAAACGTT |
| 12 | 3806 | SCNN1A | GCATAGGGAAAGCACAGGTGTCCAGGTGACAGTT |
| | | | AGAGGAACTTGGTGACAGGTGTGGGC [G,T] |
| | | | GACTCAGGCTTAGGTGTTGCTACCATCTTACTTC |
| | | | CCAGCTGGTAGTTTCTGTCCTCAAAC |
| 13 | 1756 | TPH1 | TCCCTTTGCTGAGCACCTGCATCCGAACACTCTT |
| | | | TACTTTATTTTCTTAATCTTCCATGTGTTCACCA |
| | | | ACTAGATTCTAAATTCCTTTATGTCTTCCCTTTA |
| | | | TATTCTTCGCAGCCCTTACCTAGTTACACAGGTG |
| | | | TTCATTAAGTTATTTTAGGATGCACTGGTTAGTA |
| | | | TTGGAGATTATCTGCTCTAACCTTCTCATT |
| | | | [T,G] |
| | | | TTTTAGATGAAAAAACTATAGGGTGATTAGTGCT |
| | | | CATGTTTCATTATCATGTCCCCAGCACTCTTCCT |
| | | | CAGCTACCCAAATTGGTAAGAGAACATACCCTCC |
| | | | TCCCACACTTATACACAGAGCACCCCCATCTTAG |
| | | | GCTGACTTGGGAGAGGTCAGAAGAGGAGATCTGG |
| | | | TTCAGCTGTTACTAAGATTGTAGCTTAAGA |
| 14 | 3784 | TPH1 | TTTAGTTTTTATAGAAACCACTTACATAATTTCT |
| | | | TCCCTTGTAGAAACTGTATTGCTCTTTCCTGTTT |
| | | | ATTTCTTCTAGTTTTGTGTATAGTTGTTGAATAC |
| | | | TGTTAAGGAGTTAAATAGATATGTAGATTTTTTT |
| | | | AAGTCTATGAGAGAAACTCAAAGACAGCAATTTC |
| | | | AGACCACGATGGAAATAAACTAAACTCTCTTGAG |
| | | | TCACAGCATAACTGCTTTGTTTTCTCCACTATGG |
| | | | TAATCTGCTGAGATTATCTACTGAGATAAAAGGG |
| | | | AAAATTTATACTTCTTAAATATTGTAAACCCTTG |
| | | | AAGATAGATCACTGATCATTTTACTTCCTATACT |
| | | | AGCTAGAAAAAAGTAAAGAGGCCTGGA [C,T] |
| | | | CAAACAAATATAAAGAACTAAAAGGAACAAAATT |
| | | | GTCCTTACCCTTTGTTTATGCCTCTACTAGCACC |
| | | | ATCTTTTTTCTTGCTAAAATTATAAAATCTTAAT |
| | | | GTGGAAAATGTCTTCAACCTTCTCCAAGATGTTT |
| | | | GAATCTCCTCTACCAAATAATGGGTGTGGAGCTT |
| | | | TTGCATGAACACCCCCACGGATGAGGAACTAACC |
| | | | ATCTCATCAGCCTATTCTATTTTCAAATCACTCT |
| | | | TTAACAAGATATTCTCTATTCTAGGCCAAAAGCT |
| | | | TGAAGTTTCTATCCAACAGTCCTTTACTT |

What is claimed is:

1. The use of tegaserod in the manufacture of a medicament for the treatment of chronic constipation in a selected patient population, wherein the patient population is selected on the basis of genetic polymorphisms in biomarker genes present in the patients, wherein the genetic polymorphisms are indicative of the efficacy of tegaserod in treating chronic constipation.

2. A method for determining the responsiveness of an individual with a gastrointestinal disorder to treatment with tegaserod, comprising;
(a) determining, for the two copies of the gene present in the individual, the identity of a nucleotide pair at a polymorphic genetic locus, and
(b) assigning the individual to a “high” responder group if the nucleotide pair at a polymorphic site in the region of the gene indicates that the individual is responsive to treatment with tegaserod for chronic constipation.

3. The method of claim 2, wherein the gastrointestinal disorder is selected from the group consisting of constipation-predominant irritable bowel syndrome (C-IBS), functional dyspepsia, gastroesophogeal reflux disease, and diabetic gastropathy.

4. A method for treating chronic constipation in a subject, comprising the steps of:
(a) obtaining the genotype of a subject at a genetic locus from one or more genes indicative of the indicative of efficacy of tegaserod in treating chronic constipation, wherein the genes selected from the group consisting of the gene for 5-HT₄ receptor (HTR4); the gene for 5-HT₃ receptor, subunit B (HTR3B); the gene for motilin (MLN); the gene for aquaporin 3 water channel (AQP3); the gene for solute carrier family 12, member A2 (SLC12A2), also known as the sodium/potassium/chloride cotransporter 1 (NKCC1); and the gene for non-voltage gated sodium channel, alpha subunit (SCNN1A), also know as the amiloride-sensitive epithelial sodium channel alpha subunit (ENaC alpha), and
(b) either (i) administering tegaserod to the subject if the genotype of the subject is indicative of the efficacy of indicative tegaserod in treating chronic constipation, or (ii) administering an alternative therapy to the subject if the genotype of the subject is not indicative of the efficacy of indicative tegaserod in treating chronic constipation.

5. The method of claim 4, wherein the chronic constipation is constipation-predominant irritable bowel syndrome (C-IBS).

6. The method of claim 4, wherein the gene for HTR4 has a sequence selected from the group consisting of the nucleotide sequences of SEQ ID NO: 1, SEQ ID NO:2, SEQ ID NO:3, SEQ ID NO:4, SEQ ID NO:5 and SEQ ID NO:6.

7. The method of claim 4, wherein the gene for HTR3B has a sequence selected from the group consisting of the nucleotide sequences of SEQ ID NO:7.

8. The method of claim 4, wherein the gene for MLN has a sequence selected from the group consisting of the nucleotide sequences of SEQ ID NO:8.

9. The method of claim 4, wherein the gene for AQP3 has a sequence selected from the group consisting of the nucleotide sequences of SEQ ID NO:9.

10. The method of claim 4, wherein the gene for SLC12A2 has a sequence selected from the group consisting of the nucleotide sequences of SEQ ID NO:10 and SEQ ID NO:11.

11. The method of claim 4, wherein the gene for SCNN1A has a sequence selected from the group consisting of the nucleotide sequences of SEQ ID NO:12.

12. A method for treating chronic constipation in a subject, comprising the steps of:
(a) obtaining the genotype of a subject at a genetic locus from one or more genes indicative of the indicative of efficacy of tegaserod in treating chronic constipation, wherein the genes selected from the group consisting of 5-HT₄ receptor (HTR4); the gene for 5-HT₃ receptor, subunit B (HTR3B); the gene for motilin (MLN); the gene for aquaporin 3 water channel (AQP3); the gene for solute carrier family 12, member A2 (SLC12A2), also known as the sodium/potassium/chloride cotransporter 1 (NKCC1); the gene for non-voltage gated sodium channel, alpha subunit (SCNN1A), also know as the amiloride-sensitive epithelial sodium channel alpha subunit (ENaC alpha); and the gene for tryptophan hydroxylase 1 (TPH1), and
(b) either (i) administering tegaserod to the subject if the genotype of the subject is indicative of the efficacy of indicative tegaserod in treating chronic constipation, or (ii) administering an alternative therapy to the subject if the genotype of the subject is not indicative of the efficacy of indicative tegaserod in treating chronic constipation.

13. The method of claim 12, wherein the chronic constipation is constipation-predominant irritable bowel syndrome (C-IBS).

14. The method of claim 12, wherein the gene for TPH1 has a sequence selected from the group consisting of the nucleotide sequences of SEQ ID NO:13 and SEQ ID NO:14.

15. A method for identifying an association between a haplotype of a gene and a gastrointestinal disorder trait, which comprises comparing the frequency of the haplotype in a population exhibiting the gastrointestinal disorder trait with the frequency of the haplotype in a reference population, wherein a higher frequency of the haplotype in the gastrointestinal disorder trait population than in the reference population indicates the haplotype is associated with the gastrointestinal disorder trait and wherein the gene is selected from the group consisting of 5-HT₄ receptor (HTR4); the gene for 5-HT₃ receptor, subunit B (HTR3B); the gene for motilin (MLN); the gene for aquaporin 3 water channel (AQP3); the gene for solute carrier family 12, member A2 (SLC12A2), also known as the sodium/potassium/chloride cotransporter 1 (NKCC1); the gene for non-voltage gated sodium channel, alpha subunit (SCNN1A), also know as the amiloride-sensitive epithelial sodium channel alpha subunit (ENaC alpha); and the gene for tryptophan hydroxylase 1 (TPH1).

16. Use of a gene product of a gene selected from the group consisting of the gene for 5-HT₄ receptor (HTR4); the gene for 5-HT₃ receptor, subunit B (HTR3B); the gene for motilin (MLN); the gene for aquaporin 3 water channel (AQP3); the gene for solute carrier family 12, member A2 (SLC12A2), also known as the sodium/potassium/chloride cotransporter 1 (NKCC1); the gene for non-voltage gated sodium channel, alpha subunit (SCNN1A), also know as the amiloride-sensitive epithelial sodium channel alpha subunit (ENaC alpha); and the gene for tryptophan hydroxylase 1 (TPH1) as a target for drug activity, wherein the use comprises the steps of:
(a) contacting the drug with a first gene product encoded by a polynucleotide having nucleotide pair at a polymorphic site in the region of a selected gene indicating high responsiveness to treatment with tegaserod for chronic constipation;
(b) identifying the activity of the drug on said first gene product;
(c) contacting the drug with a second gene product encoded by a polynucleotide having nucleotide pair at a polymorphic site in the region of the selected gene indicating low responsiveness to treatment with tegaserod for chronic constipation;
(d) identifying the activity of the drug on said second gene product;
(e) identifying the similarities and differences between the activity identified in step (b) and the activity identified in step (d).


SEQUENCE LISTING (14 sequences)

SEQ ID NO: 1 - DNA, 238 bp, Homo sapiens
  variation (1)...(238): SNP in gene for 5-HT4 receptor (HTR4) (PG ID #1746)
tctagataaggcgggaactgtcataagcatgactatcaaagctaaataaaacatactgaattaaatacaaaatatgaagattaagtaaaatattctttgaaagattggaaagtaaccaagacaaataagacttagtagctacactctcaaaaagaaaaagaagacctctactatttcytttggggcctttactgagtcatataaggtctgtgtaaaagtaattgtggtttttgccatt

SEQ ID NO: 2 - DNA, 601 bp, Homo sapiens
  variation (1)...(601): SNP in gene for 5-HT4 receptor (HTR4) (PG ID #3743)
aatctgctgtcaataccctaggagagggcttcctttcatcttctcctagactatcacaaccacttttatcccactaactactttgtctttgtcttcaaattcttagtatatgagacagcttaaatttaacatgttcaaaattgaactcctgatttccctaacctcaaacctgcttcccatatgaatacatagcaaatctatttctccagttgctcaggttaaaaatttggaagccacttttaccaggtctctacctcttctacctccagtacatcagcaaatcctgatagctctaacttcmtaacgtttcctgatcccaaccctttaccagccattcaatgtcataccattctagttcctactacatcatgcttcacttggtgactgcaaagtcctctcaactggcatccctgcttccatcatttctcacatacaatccactcttcacccactagacagaatgagctttgaaaaatattgagattggaacatatcacttccatactcactgcccttaagatggcttgtcatctcactaggagtaaacccagaaccctcaccatgacctatgtaaatttacaaagctggccactggctgctt

SEQ ID NO: 3 - DNA, 601 bp, Homo sapiens
  variation (1)...(601): SNP in gene for 5-HT4 receptor (HTR4) (PG ID #3747)
gggttacaggttggcagcagctgtgttcctgatctctttgtgggatctgataataactctctcccctcggcccttcaggcacaaggatgataacagctcactgctgtgctaagctccaaggtattccaccatcctttgttgattttaaccttgcacacacctttaactcatttgtgcctgctttccctttcctatgggacctcctatcaggctgctcaacctatcactgttccttcatgtcaaccacaaatttggttagccgttaaagtcttcattcaagaaatggatgtgaatatttaawgttcaaagctaagggccactgtcaatttaatccaaattgcctgttattatgacctaggctttggtgaccacagaagtatactgaggattgtgacacatacagccgattccaatcgctttcattttgggttgatttagtagggtatatctggaatatattctgtgaaggttatattttagtggagatgatcttgattctctagtaggattgaaaagaaagtgaatgaagaaaggaaagaagaaagtaagaaaagaattgagggaggaaagaaggaagagagagaaaaaagaaaaaggacct

SEQ ID NO: 4 - DNA, 601 bp, Homo sapiens
  variation (1)...(601): SNP in gene for 5-HT4 receptor (HTR4) (PG ID #3753)
cctgtgccccgacaggccatgggttagacaagtttgtcctagacccaaaccccacatttgaatatgggaaaaccagggcccagaaaagaagagctgacttgtcagagcgtgctagtggctggtctgggagaggctgcttgtcctgtgagcccaccactgagaacacacagttccaaaaatcaagaattctctgtctttcctgagctcttttgactgaaaattaaggtgcatgccctctccagatggaagcagcctgcaaagtcatggctttggccttgtccagaacttctgttaagctggyacagaagacaaggggccaaagggaatgaggaaccagtctggcagcagttggagaatgagaaagtccctccattcagctgagaagtatttttataggcagattgatagattgtttatgactgaagttcagatgaaaagaaattccagatatgcttatttaaatcacccttctacacttaagttctaactatcaacagtacaaatatcagatagggaagatttaattcaatgaggaaaactgtacacttagaaaaaagaccttaataggctgatgtggccaccaaaaattcatttgatctta

SEQ ID NO: 5 - DNA, 601 bp, Homo sapiens
  variation (1)...(601): SNP in gene for 5-HT4 receptor (HTR4) (PG ID #3754)
aaatattatgtacaaagcctgaacctgccctaaaagagtatgtgagaaggagaaacttttaatatactttccttgtgaaatgcatttcaatcctattgggaaaacaaatgtaattgtgtgacacagttcacaatgacagacacttttcatttatacattacaaaatcgaattatactccagggagatcattaagaaaatgcagatacatttatctccagcacaagattaaacatgcaaccttgaagtaagaattgtatgttttacaagtaaacctgtttaaaagaggatcggagactaagwaactgttggagaattttgccgataaaacacatcaaatttttgagctgtgcagaggaagggtgacaattcagctgggtgtatccaaaaaagaatcttgcagtactatagagaatactatcttcaaggaagtgaaattgactttttttttaaaaagggaattacatttaatgctaagtaaaggacaaagtacagcatgttcagagaaaggggataaaaatggttaaaagtctgaaaagtatggcttatgaggccagcagaaggatctagaaatatttggcctaatatagggaagacttgggg

SEQ ID NO: 6 - DNA, 601 bp, Homo sapiens
  variation (1)...(601): SNP in gene for 5-HT4 receptor (HTR4) (PG ID #3756)
tgtgtctgtatcttttctgtcactttatataactaattactgtttactctttccaagcagggtgtgtggggatccaaactgtgggagagaatggtgttactttggggaagcctttgcagaaacaagacagtctggaagaaagggaaactccagctgcagaattttcttctcttttcttgctgtggccccatcctcccacctggctctaggggaaagtcctatttaaaagtgagagttagtaacttagcatctttcagcagatatcacagttgcctctggcacagcctcttgcaccctcgtmcttcctctcctggctgtgcctgcctcagcatggggataactgactgccctcagttttcctcaccctgctactagatttgtagctggatttggggacctctgaaggaattttaaccacatagggaagcggtatgatcaagtggaagaaatgaaaacaccaaggctcgtaagattgagatctaatcctgctctgccaccaaccagctgagtctcccctgctgaaccatcagtcagctctgtggttgcaagaccgatagacttttctcagcactggtctcatttaatctctgcaacacttgca

SEQ ID NO: 7 - DNA, 601 bp, Homo sapiens
  variation (1)...(601): SNP in gene for 5-HT3 receptor, subunit B (HTR3B) (PG ID #3755)
agaagtgccatgtgttgacattatttaagatacagatctggttactaatgttgaggttactcattatgtctgtgttgttatagcagcaaatgagttaacttacgaggctgtcactgaaaagctcatctttgccagggtgaatcatctcatggaaaatgcgattctgttttgcagggctaggctggtcctggacctcatggtcactaccatctcctaatcagcctatgttttgaaatgaccaacatcctctctgtgacaacaagttctcttgtgtttcatatagtgtggacattgaaagatmccctgaccttccctatgtttatgtgaactcatctgggaccattgagaactataagcccatccaggtggtctctgcgtgcagtttagagacatatgcttttccatttgatgtccagaattgcagcctgaccttcaagagcattctgcatacaggtaaaccatgagagatacccattaatgctaggttggtgcacataggtgaaatgatattatactatccttcaggtctattttattcttgcagataattggctatttaaaaattggaatctcttcttgcggttttttggctcctgctgta

SEQ ID NO: 8 - DNA, 121 bp, Homo sapiens
  variation (1)...(121): SNP in gene for motilin (MLN) (PG ID #1783)
cattgtccagctccaagatggtatcccgtaaggctgtggctgctctgctggtggtgcatgyagctgccatgctggcctcccagacggaagccttcgtccccatcttcacctatggcgaact

SEQ ID NO: 9 - DNA, 401 bp, Homo sapiens
  variation (1)...(401): SNP in gene for aquaporin 3 water channel (AQP3) (PG ID #1838)
tgtcccgctgcggggagatgctccacatccgctaccggctgctccgacaggcgctggccgagtgcctggggaccctcatcctcgtggtgagtggagggagccggggaagcccttctctctccagcccttgcactccccaaactctcacttccccgaaggggctgtgttttccaaggtagcctggacccacctccccagctstgacccccacgcttaaccgcggaggatcaagctgacttccaaagtcctcttccccacggttctaaccccctctctgacagctccgactcttgccagaatgacagctgttactccccagtgatagtgccgatcgtttaccctcccacaagtgactcagccaacaggctgggggcagcggtcacgcttgcagtctgggacag

SEQ ID NO: 10 - DNA, 101 bp, Homo sapiens
  variation (1)...(101): SNP in gene for solute carrier family 12, member A2 (SLC12A2) (PG ID #1842)
tgctattcaagtagcaaaggaaaactactctcacaaacttcagttcaacaragaagaatcaccattaagattgagatatggaattgactaaaaccgaagtc

SEQ ID NO: 11 - DNA, 481 bp, Homo sapiens
  variation (1)...(481): SNP in gene for the non-voltage gated sodium channel, alpha subunit (SCNN1A)  (PG ID #3801)
gtcaagggtggaattgccgaggaatgttaacttaatctctcaaaagtttgtagcgggtttggctagttacgtgataccggagggctgcctctaacaaccttccccatccagttaggtatctcgtgtgcactttctttcccacccacgctcaacagtcaccayccctctgaatgacagtagtgtttgtgggccctttagaggagaatgtgcagtgaggatctctcgagagaggttggagagcacctaccatctgtgtcgttttgaaagattgtgttgtgtggcttatgtacgcctattagggatcgcaagagtgggaatgttgctgtttgaaggaaaaccttaacagggtgtgtttatggtctatgagttattaaagctcaattcttgcaatcttgaatatacaaaggatcatagagatcttgtcttgggacacgctgttcctaataatggcataactctttttttttgcgatgaaaacgtt

SEQ ID NO: 12 - DNA, 121 bp, Homo sapiens
  variation (1)...(121): SNP in gene for the non-voltage gated sodium channel, alpha subunit (SCNN1A) (PG ID #3806)
gcatagggaaagcacaggtgtccaggtgacagttagaggaacttggtgacaggtgtgggckgactcaggcttaggtgttgctaccatcttacttcccagctggtagtttctgtcctcaaac

SEQ ID NO: 13 - DNA, 401 bp, Homo sapiens
  variation (1)...(401): SNP in gene for tryptophan hydroxylase 1 (TPH1) (PG ID #1756)
tccctttgctgagcacctgcatccgaacactctttactttattttcttaatcttccatgtgttcaccaactagattctaaattcctttatgtcttccctttatattcttcgcagcccttacctagttacacaggtgttcattaagttattttaggatgcactggttagtattggagattatctgctctaaccttctcattkttttagatgaaaaaactatagggtgattagtgctcatgtttcattatcatgtccccagcactcttcctcagctacccaaattggtaagagaacataccctcctcccacacttatacacagagcacccccatcttaggctgacttgggagaggtcagaagaggagatctggttcagctgttactaagattgtagcttaaga

SEQ ID NO: 14 - DNA, 669 bp, Homo sapiens
  variation (1)...(669): SNP in gene for tryptophan hydroxylase 1 (TPH1) (PG ID #3784)
tttagtttttatagaaaccacttacataatttcttcccttgtagaaactgtattgctctttcctgtttatttcttctagttttgtgtatagttgttgaatactgttaaggagttaaatagatatgtagatttttttaagtctatgagagaaactcaaagacagcaatttcagaccacgatggaaataaactaaactctcttgagtcacagcataactgctttgttttctccactatggtaatctgctgagattatctactgagataaaagggaaaatttatacttcttaaatattgtaaacccttgaagatagatcactgatcattttacttcctatactagctagaaaaaagtaaagaggcctggaycaaacaaatataaagaactaaaaggaacaaaattgtccttaccctttgtttatgcctctactagcaccatcttttttcttgctaaaattataaaatcttaatgtggaaaatgtcttcaaccttctccaagatgtttgaatctcctctaccaaataatgggtgtggagcttttgcatgaacacccccacggatgaggaactaaccatctcatcagcctattctattttcaaatcactctttaacaagatattctctattctaggccaaaagcttgaagtttctatccaacagtcctttactt